(19) United States
(12) Patent Application Publication
(10) Pub. No.: US 2015/0099289 A1
(43) Pub. Date: Apr. 9, 2015

(54) CARBONIC ANHYDRASE XEROGEL PARTICLES

(71) Applicant: Akermin, Inc., St. Louis, MO (US)

(72) Inventors: Tracy L. Bucholz, St. Louis, MO (US); Aleksey Zaks, Hoboken, NJ (US); Dawn Powell, Edwardsville, IL (US); Luke Weber, Edwardsville, IL (US); Matthew Hulvey, Highland, IL (US); John Reardon, Lake St. Louis, MO (US); Brett Rambo, St. Charles, MO (US)

(21) Appl. No.: 14/510,807

(22) Filed: Oct. 9, 2014

Related U.S. Application Data
(60) Provisional application No. 61/889,027, filed on Oct. 9, 2013.

(51) Int. Cl. B01D 19/00; C12N 11/04

(52) U.S. Cl. CPC B01D 19/0073; C12N 11/04
USPC 435/182; 435/266

(57) ABSTRACT

The present invention generally relates to carbonic anhydrase xerogel particles comprising a polysilicate-polysilicone copolymer and carbonic anhydrase. The carbonic anhydrase xerogel particles can be used for carbon dioxide capture.

### FIELD OF THE INVENTION

The present invention generally relates to carbonic anhydrase xerogel particles comprising a polysilicate-polysilicone copolymer and carbonic anhydrase. The carbonic anhydrase xerogel particles can be used for carbon dioxide capture.

### BACKGROUND OF THE INVENTION

Technologies are being developed for capturing carbon dioxide (CO₂) from industrial gas streams to reduce energy costs and the environmental impact of CO₂ in the atmosphere. Major sources of CO₂ emissions include power plants, cement kilns, natural gas processing facilities, ammonia plants, and hydrogen plants. The captured CO₂ can be sequestered or can be reutilized for enhanced oil recovery, food processing, or accelerated algae growth that could have multiple applications. In the cases of natural gas processing and ammonia production, removal of CO₂ is a necessary step to meet product specifications. In the case of industrial hydrogen production, CO₂ removal can improve plant efficiency and increase product output.

Currently several alternative CO₂ capture technologies are in various stages of commercial practice and development. These include chemical absorption using amine solvents (particularly monoethanolamine, MEA), physical adsorption, membrane separation, cryogenic distillation, and mineral carbonation. Chemical absorption with amines is currently considered the lowest cost method of CO₂ removal for the majority of gas streams, particularly for the clean-up of low levels of CO₂ in natural gas. MEA systems are more reactive, and therefore preferred, but the energy requirements to remove the absorbed CO₂ from the MEA is very high, for example, about 4 million BTU/tonne of CO₂, and can require up to about one-third of a power plant's boiler output.

One emerging alternative to amine stripping is to incorporate biocatalysts that are specific for carbon dioxide conversion (CO₂) in the presence of low duty solvents, subsequently lowering the regeneration energy requirements and lowering overall cost. Carbonic anhydrases (CAs), EC 4.2.1.1, are a family of enzymes that are ubiquitous in nature and are known to reversibly convert bicarbonate into CO₂ and water catalytically.

There is a need in the art for improved materials, compositions, methods, processes, and systems which improve the stability and efficiency of enzymes for use in the catalysis of industrial processes.

### SUMMARY OF THE INVENTION

One of the various aspects of the invention is a carbonic anhydrase xerogel particle comprising carbonic anhydrase entrapped within a xerogel particle. The carbonic anhydrase xerogel particle being derived from a sol comprising (i) an alkoxy silane, an organotrialkoxy silane, or a metasilicate, (ii) a hydrophobic organotrialkoxy silane, a hydrophobic organohalosilane, or a combination thereof, (iii) a poly(silicone), (iv) a hydrophilic additive, and (v) carbonic anhydrase.

Another aspect of the invention is an enzyme xerogel particle comprising an enzyme entrapped within a xerogel particle. The enzyme xerogel particle being derived from a sol comprising (i) an alkoxy silane, an organotrialkoxy silane, or a metasilicate, (ii) a hydrophobic organotrialkoxy silane, a hydrophobic organohalosilane, or a combination thereof, (iii) a poly(silicone), (iv) a hydrophilic additive, and (v) an enzyme.

Another aspect is a carbonic anhydrase xerogel particle comprising carbonic anhydrase entrapped within a xerogel particle. The carbonic anhydrase xerogel particle is derived from a sol comprising (i) an alkoxy silane or an organotrialkoxy silane or metasilicate, (ii) a poly(silicone), and (iii) carbonic anhydrase wherein the carbonic anhydrase xerogel particle has a particle size range of 100 nm to less than 10 μm or more than 20 μm to 250 μm.

A further aspect is a carbonic anhydrase xerogel particle comprising carbonic anhydrase entrapped within a xerogel particle. The carbonic anhydrase xerogel particle is derived from a sol comprising (i) an alkoxy silane or an organotrialkoxy silane or metasilicate, (ii) a poly(silicone), and (iii) carbonic anhydrase wherein the carbonic anhydrase xerogel particle is further modified by a hydrophobic or hydrophilic modifier.

Yet another aspect is a process for removing CO₂ from a CO₂-containing gas, the process comprising contacting a liquid with a CO₂-containing gas; and contacting the CO₂ in the liquid with the carbonic anhydrase xerogel particle described herein to catalyze hydration of the CO₂ and form a treated liquid comprising hydrogen ions and bicarbonate ions.

A further aspect is a system for removing CO₂ from a CO₂-containing gas comprising a reaction vessel comprising a bottom portion containing a gas inlet and a liquid outlet, a top portion containing a liquid inlet and a gas outlet, and a middle portion containing a plurality of the carbonic anhydrase xerogel particle described herein, the carbonic anhydrase being capable of catalyzing hydration of CO₂ into hydrogen ions and bicarbonate ions.

Another aspect of the invention is a method for preparing carbonic anhydrase xerogel particles comprising mixing (i) an alkoxy silane or an organotrialkoxy silane or metasilicate, (ii) a poly(silicone), (iii) a hydrophilic additive, (iv) a carbonic anhydrase, and (v) solvent to form a sol; allowing the sol to form a gel; and curing the gel at a temperature from about 55° C. to about 100° C.

Other objects and features will be in part apparent and in part pointed out hereinafter.

### BRIEF DESCRIPTION OF THE DRAWINGS

FIG. 1 is a graph of the absorbance versus time for a buoyancy (floatation separation) experiment that quantifies the progress of clarification by light absorbance over time at 70° C.

FIG. 2 is a graph of the absorbance versus time for a buoyancy (floatation separation) experiment that quantifies the progress of clarification by light absorbance over time, for various solution temperatures.

FIG. 3 is a Fourier transform infrared (FTIR) image of carbonic anhydrase polysilicate-polysilicone xerogel particles (labeled “Polysilicate-Polysilicone) as compared to polydimethylsiloxane only (labeled “Silanol-Terminated PDMS”) and carbonic anhydrase only (labeled “Carbonic Anhydrase”).

FIG. 4 shows scanning electron microscope (SEM) images of representative carbonic anhydrase polysilicate-polysilicone xerogel particles.

FIG. 5 shows a weight percent loading study of a carbonic anhydrase polysilicate-polysilicone xerogel particles generated using the synthetic procedure in Example 8 compared to soluble enzyme in a batch reactor.

FIG. 6 shows a continuous flow reactor experiment conducted by flowing a 1.2 wt. % suspension of carbonic anhydrase polysilicate-polysilicone xerogel particles prepared using the synthetic method described in Example 2 over ⅛ in. Tipton ceramic spheres.

FIG. 7 shows a continuous flow reactor experiment conducted by flowing a 0.5 wt. % suspension of carbonic anhydrase polysilicate-polysilicone xerogel particles prepared using the synthetic method described in Example 9 over Sulzer Mellapak® 500X structured packing with a 2″ column diameter.

Corresponding reference characters indicate corresponding parts throughout the drawings.

### DESCRIPTION OF THE INVENTION

It has been discovered that the utilization of sol-gel processes to immobilize carbonic anhydrase in polysilicate-polysilicone derived materials results in functional materials that demonstrate excellent ability to convert CO₂ in water to bicarbonate and a proton. These materials can be used as particles in suspension. These materials provide significant benefits for use in industrial applications involving carbonic anhydrase entrapped in a polysilicate-polysilicone xerogel particle.

The polysilicate-polysilicone particles described herein provide a versatile platform for the immobilization of enzymes. This versatility is due, in part, from the high degree to which the properties of the particles can be modified based on selection of the component substituents. For example, the particle size, pore size and distribution, hydrophilicity/hydrophobicity, and enzymatic activity of the particles can be controlled by the appropriate selection of the component substituents and the selection of particular synthetic methods.

The processes described herein can be used to generate particles of uniform or distributed particle sizes, commonly referred to as xerogels, which can be used in fluidized bed reactors or column contactors. These polysilicate-polysilicone copolymer xerogels are porous particulates that display a range of particle sizes and allow high retention of the carbonic anhydrase as well as high catalytic activity.

The resulting particles can be synthesized in a single pot using aqueous or alcohol diluents.

The porous nature of the particles, which can be broadly distributed between microporous and macroporous, facilitates transport of reactants and products into and out of the polymeric structure. As a result, the three dimensional matrix is able to effectively retain the enzyme within the particle without overly restricting its activity.

Typically, an optimized xerogel particle consists of 2 to 10% immobilized enzyme by weight.

Typically, greater than 80% carbonic anhydrase retention is observed over the course of days, weeks, and months, in various solutions (pH range 10-10.5) at 45° C.

The enzymatic activity of these particles has been demonstrated in a batch reactor vessel and in a counter-current flow column.

In batch reactor studies, suspensions containing 0.2 wt. % carbonic anhydrase xerogel particles in carbonate have shown rate multipliers as high as 17. The rate multiplier is the ratio of the overall mass transfer coefficient for the carbonic anhydrase catalyzed hydration of carbon dioxide compared to the same reaction without the carbonic anhydrase catalyst.

In batch reactor studies, carbonic anhydrase entrapped in polysilicate/polysiloxane particles have shown enhanced performance over solubilized enzyme at similar loading levels.

This aforementioned enhancement phenomenon has been attributed to the low density (i.e., high void volume) of the particles and their subsequent concentration at the surface of the reaction solution. This increased surface concentration of the immobilized enzyme particles relative to free enzyme homogeneously dispersed in the liquid phase reduces the average diffusion path length of CO₂ through the liquid, contributing to higher observed activities with immobilized carbonic anhydrase (CA) xerogel particles over soluble CA at comparable enzyme loadings.

In addition to batch reactor systems, the particles were analyzed in flow-through reactors containing both random and structured packing as contactor materials. Catalytic improvements in a well dispersed flow-through suspension system as high as six fold have been observed in a packed bed reactor system.

### Polysilicate-Polysilicone Copolymers

The composition of the particles described herein comprise a polysilicate-polysilicone copolymer, which is typically derived from an alkoxy silane or an organotrialkoxy silane and a poly(silicone). Since silicates and silicones can be designed to readily form three-dimensional polymer networks in solution, they are useful in forming polymers for the entrapment of enzymes.

Generally, the encapsulation material comprising an entrapped enzyme can be prepared using a sol-gel process technique. A “sol-gel” process is one in which a colloidal composition, or “sol,” acts as the precursor for an integrated network, or “gel” of network polymers and/or discrete particles.

The polysilicate-polysilicone copolymer can be derived from reaction of a sol, the sol comprising (i) an alkoxy silane or an organotrialkoxy silane, (ii) a poly(silicone), (iii) a hydrophilic additive, and (iv) a biocatalyst that catalyzes hydration of carbon dioxide.

The sol can optionally further comprise a catalyst.

The sol can optionally further contain a surfactant.

As described in more detail below, the biocatalyst that catalyzes hydration of carbon dioxide can be a carbonic anhydrase.

The alkoxy silane can have a structure of Formula 1

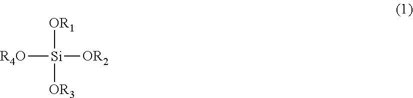

wherein R₁, R₂, R₃, and R₄ are independently hydrogen or C₁-C₄ alkyl; preferably R₁, R₂, R₃, and R₄ are independently methyl or ethyl.

Alkoxy silanes typically include tetramethylorthosilicate, tetraethylorthosilicate, methyltriethylorthosilicate, ethyltrimethylorthosilicate, dimethyldiethylorthosilicate, tetraglyceryl silicate, sodium metasilicate or a combination thereof. Tetramethylorthosilicate and tetraethylorthosilicate are especially preferred.

The organotrialkoxy silane has a structure of Formula 2

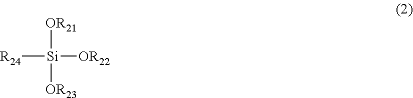

wherein R₂₁, R₂₂, R₂₃, and R₂₄ are independently hydrogen or C₁-C₄ alkyl; preferably R₂₁, R₂₂, R₂₃, and R₂₄ are independently methyl or ethyl.

The organotrialkoxy silane is typically trimethoxymethylsilane, trimethoxyethylsilane, or a combination thereof.

Hydrophilic organotrialkoxy silanes can also be utilized to improve dispersion of the particles in solution and aqueous transport. Preferred versions include 2[(methoxy(polyethyleneoxy)propyl]trimethoxy silane.

The poly(silicone) is typically selected from the group consisting of poly(siloxanes), poly(glyceryl silicates), and polysilsesquioxanes.

Preferably, the poly(silicone) species is typically silanol or alkoxy silane terminated.

Poly(siloxanes) are a preferred type of poly(silicone). Generally, poly(siloxanes) have a structure of Formula 3

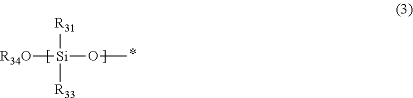

wherein R₃₁, R₃₂, R₃₃, and R₃₄ are independently hydrogen or C₁-C₄ alkyl; preferably R₃₁, R₃₂, R₃₃, and R₃₄ are independently methyl or ethyl.

Poly(siloxanes) typically include poly(dimethylsiloxane), poly(dimethylsiloxane)-co-poly(alkene oxide), poly(dimethylsiloxane)-g-poly(ethylene oxide), and block copolymers of poly(dimethylsiloxane) and poly(ethylene oxide), or combinations thereof. Preferably, the poly(siloxane) is silanol terminated. Silanol terminated poly(dimethylsiloxane) is a preferred poly(siloxane).

The silanol terminated poly(dimethylsiloxane) can have an average molecular weight of about 200 daltons, about 550 daltons, about 1100 daltons, about 2750 daltons, or about 4200 daltons. The average molecular weight can range from about 200 daltons to about 2750 daltons, from about 200 daltons to about 1100 daltons, or from about 450 daltons to about 650 daltons.

The ratio of alkoxy silane or organotrialkoxy silane to poly(silicone) is one parameter that affects the enzyme activity. For example, changing the amount of a poly(silicone) in the sol solution relative to the alkoxy silanes or organotrialkoxy silanes typically changes the pore size and volume. Particles with appropriately controlled porosity are more permeable to the reactant stream and, subsequently, allow for a higher level of retained enzyme activity.

Where a mixture of alkoxy silanes and poly(silicones) is used, the molar ratio of alkoxy silane to poly(silicone) is typically about 1:1, about 2:1, about 3:1, about 4:1, about 5:1, about 6:1, about 7:1, about 8:1, about 10:1, about 12:1, about 15:1, or about 20:1. The molar ratio can range from about 1:1 to about 20:1, from about 2:1 to about 8:1, or from about 3:1 to about 5:1.

When the poly(silicone) is a poly(siloxane), the molar ratio of alkoxy silane to poly(siloxane) is the same as the alkoxy silane to poly(silicone) ratios described above.

The sol used to prepare the particles can also contain a hydrophilic additive.

If prepared in the absence of hydrophilic additives, the polysilicate-polysilicone particles described herein are more hydrophobic. This is acceptable for some applications, particularly for applications in non-aqueous environments. In other applications, however, a high degree of hydrophobicity can result in significantly reduced enzyme activity by retarding the transport of polar reactants and products to and from the encapsulated enzyme. It is therefore desirable to incorporate a hydrophilic additive into the particle composition, particularly where the enzyme is used to catalyze hydrophilic reactants in an aqueous environment.

Examples of typical hydrophilic additives include poly(vinyl alcohol), poly(ethylene oxide), a quaternary ammonium polymer, a crown ether, a cyclodextrin, a surfactant, poly(l-methyl-4-vinylpyridinium bromide), poly(acrylamide-methacryloxyethyltrimethylammonium bromide), and a combination thereof.

Suitable quaternary ammonium polymers can include, for example, poly(diallyldimethylammonium chloride), poly(l-methyl-4-vinylpyridinium bromide), poly(acrylamide-methacryloxyethyltrimethylammonium bromide), or a combination thereof. Preferably, the quaternary ammonium polymer comprises poly(diallyldimethylammonium chloride).

Cyclodextrin additives can include, for example, α-cyclodextrin, β-cyclodextrin, γ-cyclodextrin, and combinations thereof. β-Cyclodextrin is preferred.

Crown ethers are a preferred class of hydrophilic additives. Suitable crown ethers include 12-crown-4,1,7-diaza-12-crown-4; 1,4,8,11-tetrathiacyclotetradecane; 1,4,8,12-tetraazacyclopentadecane; 15-crown-5,18-crown-6; dibenzo-18-crown-6; dicyclohexano-18-crown-6; (18-crown-6)-2,3,11,12-tetracarboxylic acid; 1-aza-18-crown-6; diaza-18-crown-6; and a combination thereof. A crown ether comprising 18-crown-6 is especially preferred.

Preferably, the particles are derived from a sol comprising tetramethylorthosilicate, polydimethylsiloxane, a crown ether, and a carbonic anhydrase.

The molar ratio of alkoxy silane and/or organotrialkoxy silane to hydrophilic additive is typically about 48:1, about 36:1, about 24:1, about 12:1, or about 4:1. The ratio can range from about 4:1 to about 60:1, from about 15:1 to about 45:1, or from about 30:1 to about 40:1.

The molar ratio of poly(silicone) to hydrophilic additive is typically about 12:1, about 9:1, about 6:1, about 3:1, or about 1:1. The molar ratio can range from about 1:1 to about 30:1, from about 2:1 to about 20:1, from about 5:1 to about 15:1, from about 6:1 to about 12:1, or from about 8:1 to about 10:1.

Further, the molar ratio of poly(siloxane) to hydrophilic additive is typically about 28:1, about 14:1, about 12:1, about 9:1, about 7:1, about 6:1, about 3:1, or about 1:1. The molar ratio can range from about 1:1 to about 30:1, from about 2:1 to about 20:1, from about 3:1 to about 20:1, from about 10:1 to about 20:1, from about 5:1 to about 15:1, from about 6:1 to about 12:1, or from about 8:1 to about 10:1.

The sol used to prepare the particles can also contain a hydrophobic additive.

Further, for use in a carbon capture system, hydrophobic components such as organotrialkoxy silanes can be added to the sol prior to gelation to increase the hydrophobicity of the particles. Increasing their hydrophobicity affects how these particles disperse in an aqueous environment and tends to concentrate them at the gas-liquid interface. Examples of typical hydrophobic additives include methyltrimethoxysilane, propyltrimethoxysilane, butyltrimethoxysilane, phenyltrimethoxysilane, octyltrimethoxysilane, and combinations thereof.

The sol used to prepare the particles can also contain a surfactant.

The surfactant can act as an emulsifier to more homogeneously distribute the enzyme into the monomer mixture prior to gelation. Further it can be used to control the resulting properties of the particles; including particle size, pore structure, and pore size distribution.

Surfactants suitable for use as additives include N,N-bis(3-D-gluconamidopropyl)cholamide (BigCHAP), N,N-bis(3-D-gluconamidopropyl)deoxycholamide (DeoxyBigCHAP), a polyoxyethylene alcohol (BRIJ 35 and BRIJ 58 P), 2-cyclohexylmethyl-β-D-maltoside (CYMAL-1), 2-cyclohexylethyl-β-D-maltoside (CYMAL-2), cyclohexylpentyl-β-D-maltoside (CYMAL-5), cyclohexylhexyl-β-D-maltoside (CYMAL-6), decyl-β-D-maltopyranoside, n-dodecyl-β-D-maltoside, n-hexadecyl-β-D-maltoside, undecyl-β-D-maltoside, decyl-β-D-1-thiomaltopyranoside, octyl-β-D-thioglucopyranoside, digitonin, dimethydecylphosphine oxide, dodecyldimethylphosphine oxide, (octylphenoxy)polyethoxyethanol (IGEPAL® CA630), N-octanoyl-N-methylglucamine (MEGA-8), N-nonanoyl-N-methylglucamine (MEGA-9), N-decanoyl-N-methylglucamine (MEGA-10), a polyoxy ethylene octyl phenol (NONIDET® P40-substitute), a polyoxyethylene-polyoxypropylene block co-polymer (PLURONIC® F-68), poly(ethylene glycol)-block-poly(propylene glycol)-block-poly(ethylene glycol) (PLURONIC® P-123) saponin, polyoxyethylene 9-lauryl ether (THESIT®), a polyoxy ethylene octyl phenol (e.g., TRITON® X-100 and TRITON® X-114), a polyoxyethylene derivative of sorbitan monolaurate (e.g., TWEEN® 20, TWEEN® 40, and TWEEN® 80), N,N-dimethyldodecylamine-N-oxide, hexadecyltrimethylammonium bromide (CTAB), an alcohol ethoxylate (SYNPERONIC® A7), and combinations thereof.

A preferred surfactant species is cetyl trimethylammonium bromide. Polyoxyethylene derivatives of sorbitan monolaurate are also preferred.

The molar ratio of alkoxy silane and/or organotrialkoxy silane to surfactant is typically about 130:1, about 65:1, about 32:1, about 16:1, or about 8:1. The ratio can range from about 4:1 to about 200:1, from about 40:1 to about 80:1, or from about 50:1 to about 70:1.

The particles can be derived from the reaction of various components in a sol. Typically, the sol comprises an alkoxy silane and/or an organotrialkoxy silane, a poly(silicone), and carbonic anhydrase dispersed throughout a solvent. The alkoxy silane and/or organotrialkoxy silane, and poly(silicone) undergo hydrolysis and subsequent condensation, thereby incorporating the enzyme into a gel-like material. The gel can be formed into particles. The result is a three-dimensional network polymer, wherein the enzyme molecules are encapsulated in the pores of the polymeric structure.

Particles produced according to this process typically have advantageous properties. For example, the polymeric structure can act to stabilize the enzyme against thermal and chemical damage, while possessing pore sizes sufficient for the immobilized enzyme to retain a significant portion of its catalytic activity.

The sol can further comprise a catalyst that can assist with a polymerization reaction. The catalyst can comprise ammonium fluoride, sodium fluoride, tetrabutylammonium fluoride, ammonium hydroxide, sodium hydroxide, potassium hydroxide, sodium hydroxide, or a combination thereof. A catalyst comprising ammonium fluoride is preferred.

More generally, the catalyst may comprise an acid or a base, including Lewis acids and Lewis bases. The catalyst may be used to initiate acid- or base-induced hydrolysis or condensation.

Post modification of the surface characteristics of xerogel particles after formation enables fine tuning of the homogeneity of the xerogel suspension in a wide variety of solvents without affecting particle size and porosity.

Manipulating the hydrophilicity/hydrophobicity of the xerogel particles' surface affects how these particles disperse in solution.

Optionally, surface hydroxyl groups of the polysilicate-polysilicone particles can be reacted with alkyl chlorosilanes or alkyl trimethoxysilanes with the desired hydrophobicity/hydrophilicity to form carbonic anhydrase xerogel particles having the desired hydrophilicity and/or hydrophobicity.

Hydrophilic modifiers can include, for example, methoxyethoxyundecyltrichlorosilane, methoxy(polyethyleneoxy)propyltrimethoxysilane, or a combination thereof.

Suitable hydrophobic modifiers can include, for example, an alkyl chlorosilane, a fluoroalkyl chlorosilane, a phenyl chlorosilane, an alkyl trialkoxysilane, a fluoroalkyl trialkoxysilane, a phenyl trialkoxysilane, or a combination thereof.

A hydrophobic alkyl/phenyl chlorosilane can be octadecyldimethylchlorosilane, benzyldimethylchlorosilane, nonafluorohexyldimethylchlorosilane, or a combination thereof. The hydrophobic alkyl/phenyl trialkoxysilane can be octadecyltrimethoxysilane, phenylethyltrimethoxysilane, nonafluorohexyltrimethoxysilane, or a combination thereof.

Fluoroalkyl chlorosilanes are a preferred class of hydrophobic surface modifier when xerogel particles are suspended in a carbonate solvent. Suitable fluoroalkyl surface modifiers include (3,3,3-trifluoropropyl)dimethylchlorosilane, nonafluorohexyldimethylchlorosilane, tridecafluoro-1,1,2,2-tetrahydrooctyl)dimethylchlorosilane, (heptadecafluoro-1,1,2,2-tatrahydrodecyl)dimethylchlorosilane, and a combination thereof.

Further, the biocatalyst can be a carbonic anhydrase xerogel particle comprising carbonic anhydrase entrapped within a xerogel particle, the carbonic anhydrase xerogel particle being derived from a sol comprising (i) an alkoxy silane, an organotrialkoxy silane, or a metasilicate, (ii) a hydrophobic organotrialkoxy silane, a hydrophobic organohalosilane, or a combination thereof, (iii) a poly(silicone), (iv) a hydrophilic additive, and (v) carbonic anhydrase.

Further, the carbonic anhydrase xerogel particle can have the hydrophobic organotrialkoxy silane or hydrophobic organohalosilane comprise an alkyl chlorosilane, a fluoroalkyl chlorosilane, a phenyl chlorosilane, an alkyl trialkoxysilane, a fluoroalkyl trialkoxysilane, a phenyl trialkoxysilane, or a combination thereof.

Additionally, the carbonic anhydrase xerogel particle can have the hydrophobic organotrialkoxy silane or hydrophobic organohalosilane comprise octadecyldimethylchlorosilane, benzyldimethyl chlorosilane, octadecyl trimethoxysilane, phenylethyl trimethoxysilane, nonafluorohexyl trimethoxysilane, (3,3,3-trifluoropropyl)dimethyl chlorosilane, isobutyl trimethoxysilane, nonafluorohexyldimethyl chlorosilane, tridecafluoro-1,1,2,2-tetrahydrooctyl)dimethyl chlorosilane, (heptadecafluoro-1,1,2,2-tatrahydrodecyl)dimethyl chlorosilane, or a combination thereof.

Also, the carbonic anhydrase xerogel particle can have the hydrophobic organotrialkoxy silane comprise nonafluorohexyl trimethoxysilane, isobutyl trimethoxysilane, or a combination thereof.

Further, the carbonic anhydrase xerogel particle can have the alkoxy silane comprise tetramethylorthosilicate, tetraethylorthosilicate, methyltriethylorthosilicate, ethyltrimethylorthosilicate, dimethyldiethylorthosilicate, tetraglyceryl silicate, or a combination thereof.

The carbonic anhydrase xerogel particle can have the organotrialkoxy silane be trimethoxymethylsilane, trimethoxyethylsilane, or a combination thereof.

Also, the carbonic anhydrase xerogel particle can have the alkoxy silane comprise tetramethylorthosilicate.

The carbonic anhydrase xerogel particle can have the poly(silicone) be a poly(siloxane) selected from the group consisting of poly(dimethylsiloxane), poly(dimethylsiloxane)-co-poly(alkene oxide), or a combination thereof.

The carbonic anhydrase xerogel particle can have the poly(siloxane) comprise polydimethylsiloxane.

The carbonic anhydrase xerogel particle can have the poly(silicone) be silanol-terminated.

The carbonic anhydrase xerogel particle can comprise an alkoxysilane of tetramethyl orthosilicate, a poly(silicone) of a silanol-terminated poly(dimethyl siloxane), a hydrophilic additive of a crown ether, and a hydrophobic organotrialkoxy silane of isobutyl trimethoxysilane, nonafluorohexyl trimethoxysilane, or a combination thereof.

Typically, the particles have a surface area of at least about 1 m²/g, at least 10 m²/g, at least about 20 m²/g, at least about 30 m²/g, at least about 40 m²/g, at least about 50 m²/g, at least about 60 m²/g, at least about 70 m²/g, at least about 80 m²/g, at least about 90 m²/g, at least about 100 m²/g, at least about 150 m²/g, at least about 200 m²/g, or at least about 300 m²/g.

The surface area of the particles typically ranges from about 1 m²/g to about 400 m²/g, from about 5 m²/g to about 300 m²/g, from about 10 to about 150 m²/g, or from about 15 to about 100 m²/g.

The particles typically have a pore diameter of from about 1 nm to about 200 nm, more preferably from about 2 nm to about 80 nm, more preferably from about 20 nm to about 80 nm.

The particles typically have an overall pore volume of at least about 3 μL/g to 500 μL/g. Typically, at least about 20%, at least about 30%, at least about 40%, at least about 50%, at least about 60%, at least about 70%, or at least about 80% of the overall pore volume may be attributed to pores having a pore diameter of between about 20 nm and about 80 nm.

The particles typically have a size from about 100 nm to about 500 μm, more preferably from about 500 nm to 250 μm, more preferably from about 500 nm to 10 μm.

The most advantageous particle size depends on the particular application and the system requirements for the particles. For instance, in most cases in order to maximize the surface area for contact of the carbonic anhydrase with carbon dioxide, the smallest particles possible are desired. For example, the carbonic anhydrase xerogel particles can be from about 100 nm to less than 10 μm, from about 500 nm to about 8 μm, or from about 1 μm to about 5 μm.

When the system for removing carbon dioxide includes a separation apparatus, it is advantageous for the carbonic anhydrase xerogel particles to be of sufficient size to be easily separated from the absorption liquid in the system. For example, the carbonic anhydrase xerogel particles can be from more than 20 μm to about 250 μm, from about 25 μm to about 200 μm, or from about 25 μm to about 150 μm.

For some applications, the particles typically range in size from hundreds of nanometers to hundreds of micrometers. The particles used in the suspension application described herein can range in size from 1 to 25 μm, 25 to 50 μm, 50 to 75 μm, or 100 to 250 μm.

For example, carbonic anhydrases encapsulated by the methods described herein exhibit catalytic rates equivalent to or greater than soluble enzyme in a batch reactor process.

The particles also include a catalyst that catalyzes hydration of carbon dioxide. Typically, the catalyst can be a biocatalyst such as an enzyme, a ribozyme, a deoxyribozyme, an enzyme mimic, or an organic or inorganic compound that can catalyze hydration of carbon dioxide.

Preferably, the biocatalyst is a carbonic anhydrase.

The molar ratio of alkoxy silane and/or organotrialkoxy silane to carbonic anhydrase is typically about 1300:1. The ratio can range from about 4000:1 to about 600:1.

The molar ratio of poly(silicone) or poly(siloxane) to carbonic anhydrase is typically about 325:1. The molar ratio can range from about 1000:1 to about 160:1.

Another aspect of the invention is an enzyme xerogel particle comprising an enzyme entrapped within a xerogel particle. The enzyme xerogel particle being derived from a sol comprising (i) an alkoxy silane, an organotrialkoxy silane, or a metasilicate, (ii) a hydrophobic organotrialkoxy silane, a hydrophobic organohalosilane, or a combination thereof, (iii) a poly(silicone), (iv) a hydrophilic additive, and (v) an enzyme.

Typically, the enzyme is encapsulated within the pores of the three-dimensional polysilicate-polysilicone copolymer network.

The enzyme can comprise a lipase, a glucose isomerase, a nitrilase, a glucose oxidase, a protease, a carbonic anhydrase, a pepsin, an amylase, a fungal amylase, a maltogenic amylase, a cellulase, a lactase, an esterase, a carbohydrase, a hemicellulase, a pentosanase, a xylanase, a pullulanase, a β-glucanase, an acetolactate decarboxylase, a β-glucosidase, a glutaminase, a penicillin acylase, a chloroperoxidase, an aspartic β-decarboxylase, a cyclodextrin glycosyltransferase, a subtilisin, an aminoacylase, an alcohol dehydrogenase, an amino acid oxidase, a phospholipase, a urease, a cholesterase, a desulfinase, a lignin peroxidase, a pectinase, an oxidoreductase, a dextranase, a glucosidase, a galactosidase, a glucoamylase, a maltase, a sucrase, an invertase, a naringanase, a bromelain, a ficin, a papain, a pepsin, a peptidase, a chymosin, a thermolysin, a trypsin, a triglyceridase, a pregastric esterase, a phosphatase, a phytase, an amidase, a glutaminase, a lysozyme, a catalase, a dehydrogenase, a peroxidase, a lyase, a fumarase, a histadase, an aminotransferase, a ligase, a cyclase, a racemase, a mutase, an oxidase, a reductase, a ligninase, a laccase, a chloroperoxidase, a haloperoxidase, a hydrogenase, a nitrogenase, an oxynitrilase, or combinations thereof.

When the copolymer network contains an enzyme; naturally-occurring enzymes, recombinant enzymes, artificial enzymes and modified naturally-occurring enzymes can be utilized. In addition, engineered enzymes that have been engineered by natural or directed evolution can be used. Also, an organic or inorganic molecules that mimics an enzyme's properties can be used.

Preferably, the biocatalyst that catalyzes hydration of carbon dioxide or the enzyme encapsulated is a carbonic anhydrase. The carbonic anhydrase (CA) used in the systems described herein catalyzes the reversible conversion of carbon dioxide and water to bicarbonate and a proton.

Compounds that mimic the active site of carbonic anhydrase can also be used. For example, various metal complexes have been used to mimic the carbonic anhydrase active site. For example, [Zn₂(3,6,9,12,20,23,26,29-octaazatricyclo[29.3.1.1^{14,18}]hexatriaconta-1(34), 14,16,18(36),31(35),32-hexaene)(CO₃)]Br₂.7H₂O and [Zn₂(3,6,9,12,20,23,26,29-octaazatricyclo[29.3.1.1^{14,18}]hexatriaconta-1(34), 14,16,18(36),31(35),32-hexaene)(CO₃)]Br₂.0.5CH₃COCH₃.5H₂O (See Qi et al., Inorganic Chemistry Communications 2008, 11, 929-934). Also used as a mimic for carbonic anhydrase was [tris(2-benzimidazolylmethyl)amineZn(OH)₂]²⁺, [tris(2-benzimidazolyl)amineZn(OH)₂](ClO₄)₂, and [tris(hydroxy-2-benzimidazolylmethyl)amineZn(OH)]ClO₄.1.5H₂O were also used to hydrate CO₂. (See Nakata et al., The Chemistry Letters, 1997, 991-992 and Echizen et al., Journal of Inorganic Biochemistry 2004, 98, 1347-1360).

For purposes of the present invention, an enzyme is “stabilized” if the rate of activity loss is less than the rate of activity loss seen in a non-encapsulated enzyme under the same conditions. The encapsulation of the enzyme provides a significant advantage in stability. The enzyme activity can be measured by a means that demonstrate enzyme-mediated generation of product. The activity can be followed by chemiluminescence, electrochemical, mass spectrometry, spectrophotometric (i.e. UV-Vis), radiochemical, or fluorescence assays wherein the intensity of the property is measured at an initial time and then monitored for the duration of the experiment.

The carbonic anhydrase can retain at least about 10% or more of its initial activity while the enzyme is continuously catalyzing a chemical transformation. Further, the carbonic anhydrase can retain at least about 20%, 40%, 60%, 80%, 100% or more of its initial activity while the enzyme is continuously catalyzing a chemical transformation.

With respect to the stabilization of the enzyme, the particles (i.e., enzyme encapsulation material) provide a protective environment to prevent or impede enzyme denaturation or inactivation/inhibition. To this end, it is believed that the enzyme encapsulation material physically confines the enzyme, preventing the enzyme from unfolding. The process of unfolding an enzyme from a folded three-dimensional structure is one mechanism of enzyme denaturation. The presence of specifically selected additives in the formulation also further protects the enzyme against inactivation.

A carbonic anhydrase xerogel particle having greater temperature or pH stability can also retain at least about 75% of its initial catalytic activity for at least about 10 days when actively catalyzing a chemical transformation at about 40° C. to about 70° C. as described above.

The enzyme is protected by the encapsulation material when at least 50%, 60%, 70%, 80%, or more of the carbonic anhydrase is retained in the polysilicate-polysilicone copolymer particles for at least 5, 10, 20, 40, 60, 80, 100, 150, 200, 250, 300, 350, or more days. Further, the carbonic anhydrase is retained by the immobilization material for from 5-365, 10-365, 20-365, 40-365, 60-365, 80-365, 100-365, 150-365, 200-365, 250-365, 300-365, or 350-365 days.

### Methods of Preparation

The sol is described herein above.

The gel product can be used to create discrete xerogel particles via several pathways: drying and curing of the bulk gel product and subsequently mechanically grinding and sieving to an appropriate particle size, spray drying of the gel product, or creation of the gel product in a microemulsion by dispersing the sol in a non-solvent prior to catalyst addition.

Typically, an aqueous stock solution comprising the carbonic anhydrase is prepared separately, and subsequently mixed with a second formulation comprising an organic solution of the silicate and silicone monomers and optional additives.

The carbonic anhydrase is typically prepared in a buffered solution (e.g., phosphate buffer) in which pH values of the stock solution can range from about 6 to about 10. Typically, a pH between about 6 and about 8 is preferred.

The carbonic anhydrase is mixed with a sufficient quantity of water to fully dissolve the enzyme. The enzyme stock solution typically comprises about 50 mg/mL, about 100 mg/mL, about 150 mg/mL, about 200 mg/mL, or about 300 mg/mL of the enzyme. The concentration of enzyme in the stock solution typically ranges from about 50 to about 200 mg/mL. A surfactant can be added to the enzyme solution to improve the dispersion of the aqueous phase into the organic phase during gelation.

If a carbonic anhydrase stock solution is separately prepared, it should be combined with the second formulation comprising the alkoxy silane and/or organotrialkoxy silane, and poly(silicone) species and optional additives and mixed until a finely dispersed emulsion or homogeneous mixture is achieved. The mixing preferably occurs under moderate to high shear, and can be conducted using any conventional mixing apparatus known in the art. Non-limiting examples of possible mixing apparatus include mechanical agitators, static agitators, rotating tank agitators, sonicators, and high pressure homogenizers. The mixing can occur as part of a batch, semi-batch, or continuous process.

A second solution, which comprises the silicate and silicone monomers and optional additives, may be prepared neat or in a dilute alcoholic solution. Methanol or ethanol is preferred for this purpose.

Several different methods can be used for combining the aqueous solution containing carbonic anhydrase and the organic mixture containing monomers. These include addition of the organic monomeric species to the aqueous enzyme solution under vigorous mixing or addition of the aqueous enzyme solution to a mixture of monomers, or partially hydrolyzed monomers. Once the two phases are sufficiently dispersed, a catalyst, preferably ammonium fluoride, can then be added to create the gel product.

An alternative method for diluting the gel product to enable subsequent processing into xerogel particles is to add the diluent after the catalyst addition to the carbonic anhydrase and neat monomer solution. The diluent can be an alcohol, in which ethanol is preferred.

The diluent can also be an aqueous solution of various buffers, including sodium phosphate, potassium phosphate, potassium carbonate/bicarbonate, tris-sulfate, tris-hydrochloride, with a pH from 7-10 and a concentration from about 10 mM to 200 mM. When a buffered solution is used as the diluent, it can be combined with the catalyst solution and added at the same time. The preferred buffer is potassium phosphate, pH ˜7.2, with a concentration of 25-100 mM.

Typically, the amount of sol to diluent is at least about 1 wt. %, at least about 10 wt. %, at least about 20 wt. %, at least about 30 wt. %, at least about 40 wt. %, or at least about 50 wt. %. The amount of sol to diluent can range from about 1 wt. % to about 50 wt. %, from about 1 wt. % to about 20 wt. %, or from about 1 wt. % to about 10 wt. %.

The method for preparing an immobilized enzyme can comprise mixing (i) an alkoxy silane or an organotrialkoxy silane or metasilicate, (ii) a poly(silicone), (iii) a hydrophilic additive, (iv) a carbonic anhydrase, and (v) solvent to form a sol; optionally, contacting the sol with a catalyst; forming a gel; and curing the gel at a temperature from about 55° C. to about 100° C.

The gel can be cured at a temperature from about 75° C. to about 100° C. for from about 48 hours to about 144 hours. The gel can be cured at a temperature from about 80° C. to about 100° C. from about 48 hours to about 144 hours. Preferably, the gel can be cured at about 85° C. for about 72 hours.

Typically, the xerogel particles are cured at a temperature between about 55° C. and 100° C. A curing temperature of 75° C. to 95° C. is preferred.

The curing time can range from about 24 to about 96 hours, depending on the curing temperature and the composition of the sol. Alternatively, a gradual ramping from 55° C. to 75° C. can be utilized. Preferred methods include 24 hours at 55° C. followed by 72 hours at 85° C.

To create discrete xerogel particles, the sol can be allowed to gel, then the bulk gel can be dried, cured, milled, and sieved into powders of a specific size.

The particles can also be generated by spray drying the gel product directly and curing.

A third technique to create xerogel particles involves dispersing the sol into a non-solvent prior to catalyst addition and having the gel form in this microemulsion environment. The gel particles can then be filtered off and cured to create xerogel particles.

Typically, after curing the xerogel particles are washed and hydrated in appropriate solvents. The pH of these solvents can range from 7 to 11. The particles can be hydrated in the solution that is used in the reactor for the hydration of carbon dioxide. For example, these solutions are typically potassium carbonate or potassium dimethylglycinate (KDMG).

After hydrating for a suitable amount of time and washing to remove any leached enzyme, the particles can be resuspended in the desired solvent. Typically, either carbonate or KDMG is used. Typically, suspensions containing different weight percent of solid particles can be prepared for CO₂ capture. The desired weight percent is typically dependent on the type of contactor used. Weight percent suspensions ranging from 0.05 to 20 wt. % can be prepared.

Once the particles are added to the solvent, a variety of techniques can be used to distribute the particles and achieve a well dispersed solution. These include shaking, mechanical mixing, vortexing, sonication, or a combination thereof.

### Processes and Systems for Removal of Carbon Dioxide

An encapsulated carbonic anhydrase wherein a carbonic anhydrase is encapsulated within a polysilicate-polysilicone copolymer derived particle can be used to catalyze a process for removing CO₂ from a CO₂-containing gas.

The biocatalyst particles, methods, and systems described herein are particularly useful for the capture and sequestration of carbon dioxide in a liquid environment.

Further, the biocatalyst particles, methods, and systems described herein can specifically be used for capture and sequestration of carbon dioxide in an aqueous environment.

The xerogel particles described herein comprise a polysilicate-polysilicone copolymer, a hydrophilic additive and a biocatalyst entrapped in the particle composition that catalyzes hydration of carbon dioxide. Suspensions of the resulting materials are suitable for use in industrial CO₂ stripping processes.

Generally, the process comprises contacting a liquid with a CO₂-containing gas to promote diffusion of the CO₂ into the liquid in the presence of an encapsulated enzyme to catalyze hydration of the CO₂, thereby forming a treated liquid comprising hydrogen ions and bicarbonate ions. Hydrogen ions can also combine with a base already present in solution.

The process can also comprise contacting a liquid with a CO₂-containing gas; and contacting the CO₂ in the liquid with the encapsulated enzyme described above to catalyze hydration of the CO₂ and form a treated liquid comprising hydrogen ions and bicarbonate ions.

The process can also comprise contacting a liquid with a CO₂-containing gas; and contacting the CO₂ in the liquid with particles of the polysilicate-polysilicone copolymer xerogel described above to catalyze hydration of the CO₂ and form a treated liquid comprising hydrogen ions and bicarbonate ions.

Generally, the enzyme can catalyze the hydration reaction that is the first step of a two-step sequence:

CO₂+H₂O→H⁺+HCO₃⁻ (1)

B⁻+H⁺→BH (2)

By using carbonic anhydrase to catalyze CO₂ hydration [reaction (1)], the rate of conversion of CO₂ into the bicarbonate form is accelerated. B represents a base that is present in the solution to act as a proton acceptor.

Carbonic anhydrase can also be used to catalyze the dehydration of the bicarbonate back into a base, CO₂, and water. The base can be recycled back to the first reactor where the hydration of CO₂ occurs. For example, the chemistry for dehydration of potassium bicarbonate is as follows:

2KHCO₃→K₂CO₃+H₂O+CO₂ (3)

Upon heating, bicarbonate releases the CO₂ and water and forms carbonate ions that can be recycled to the hydration reaction. The CA increases the rate of the dehydration reaction.

The carbonic anhydrase can also be used to accelerate the capture of carbon dioxide in solutions of amines, which often enable high loading capacity of CO₂ in the solvent. Preferably, the amine species is selected from a tertiary amine and/or hindered secondary amine. These amine solvents exhibit kinetic rates that are slow enough to be enhanced by the introduction of carbonic anhydrase.

Enzyme containing particles, wherein a carbonic anhydrase is encapsulated within a polysilicate-polysilicone copolymer, or polysilicate-polysilicone particles, can be used to catalyze a process for removing CO₂ from a CO₂-containing gas.

Typically, the process comprises contacting a liquid containing a suspension of enzyme containing particles with a CO₂-containing gas. An appropriate contactor is chosen to promote diffusion of the CO₂ into the liquid. As the CO₂ diffuses into the liquid it comes into contact with the enzyme-containing particles, which catalyze hydration of the CO₂, thereby forming a treated liquid comprising hydrogen ions and bicarbonate ions.

Typical reactors for utilizing a suspension derived approach include batch reactors, semi-batch, and continuous flow reactors such as packed columns with random packing or structured packing, and tray contactors.

The system used to hydrate carbon dioxide gas in a gas stream to form bicarbonate ions can use a variety of reactors, including continuous stirred tank reactors co-current flow columns, and counter-current flow column. For example, in a co-current system, the gas and liquid streams could enter the reactor in the form of microbubbles of gas in the liquid stream. In a counter-current column gas and liquid flow in opposite directions through a column containing an appropriate contactor packing material.

Alternatively, the liquid stream can contain a suspension of polysilicate-polysilicone particles containing encapsulated carbonic anhydrase as described above.

In another example, a semi-batch continuous flow system can be used to remove CO₂ gas from a mixed stream. A mixture containing CO₂ gas enters the tank wherein CO₂ is absorbed into the liquid which can contain a suspension of encapsulated enzyme. The encapsulated enzymes can further be in the form of particles. As a result, the liquid gets enriched in bicarbonate upon CO₂ conversion and subsequently the treated gas stream with depleted CO₂ levels continuously flows from the tank.

The batch system can include a suspension of entrapped enzyme particles wherein the enzyme is carbonic anhydrase and the encapsulation material comprises a polysilicate-polysilicone copolymer described in more detail herein. Thus, the semi-batch continuous flow system described above can contain a suspension of encapsulated carbonic anhydrase particles of the particle size described herein wherein the carbonic anhydrase is entrapped in a polysilicate-polysilicone copolymer.

The carbonic anhydrase can also be encapsulated in a material derived from reaction of a sol, the sol comprising (i) an alkoxy silane or an organotrialkoxy silane or metasilicate, (ii) a poly(silicone), and (iii) a carbonic anhydrase.

The stripper can optionally have carbonic anhydrase encapsulated in particles flowing in a bicarbonate-rich solution over standard reactor packing materials. Rates of this reaction to produce CO₂ can be increased by adding heat, and the removal of CO₂ from the stripper could be increased by operating below atmospheric pressure.

These system designs can be combined in different configurations depending on the specific application or gas stream to be treated. For example, the system specifications can be tailored to the CO₂ content of the feed stream and the overall purity, recovery, and contaminant levels required for the product streams along with the temperature and pressure requirements of both streams. The use of encapsulated enzymes increases the range of system operating conditions and reduces the reactor sizes as compared to the non-catalyzed systems.

Also, the system design can comprise a standard absorption unit and a stripping (reactive distillation) unit. The core components of the standard carbon capture system (CCS) are an absorbing unit operation, a stripping unit operation, and a heat exchange component between the two unit operations. Peripheral equipment could include standard control hardware and software, flow monitoring and regulation (e.g., control valves, flow meters), pumps, pH monitoring (e.g., pH meters), temperature monitoring (e.g., temperature monitors), or any combination thereof. The additional equipment could provide means for monitoring and controlling the process.

The system can comprise a plurality of reaction vessels, wherein two or more reaction vessels contain the encapsulated carbonic anhydrase.

As described above, the liquid is contacted with the CO₂-containing gas to help absorb the CO₂ and increase the CO₂ concentration in the liquid.

Preferably, the liquid comprises an organic or inorganic base. The base is a proton acceptor.

The base can be an ammonium hydroxide, a metal carbonate, a quaternary ammonium carbonate, a quaternary ammonium alkoxide, an amine (cyclic, primary, secondary, and tertiary), an amino acid, an alkanolamine, or a combination thereof.

The metal carbonate can be lithium carbonate, sodium carbonate, potassium carbonate, rubidium carbonate, cesium carbonate, magnesium carbonate, calcium carbonate, strontium carbonate, barium carbonate, ammonium carbonate, a carbonate salt of an organic cation, or a combination thereof. For example, the carbonate salt of an organic cation can be a tetraalkyleammonium carbonate (e.g., tetramethylammonium carbonate, tetraethylammonium carbonate, tetrapropylammonium carbonate, tetrabutylammonium carbonate, tetrapentylammonium carbonate, or tetrahexylammonium carbonate) an alkyltrimethyl ammonium carbonate (e.g., ethyltrimethyl ammonium carbonate, propyltrimethyl ammonium carbonate, butyltrimethyl ammonium carbonate, pentyltrimethyl ammonium carbonate, hexyltrimethyl ammonium carbonate, hepyltrimethyl ammonium carbonate, octyltrimethyl ammonium carbonate, nonyltrimethyl ammonium carbonate, decyltrimethyl ammonium carbonate, dodecyltrimethyl ammonium carbonate, or undecyltrimethyl ammonium carbonate), an alkyltriethylammonium carbonate (e.g., methyltriethyl ammonium carbonate, propyltriethyl ammonium carbonate, butyltriethyl ammonium carbonate, pentyltriethyl ammonium carbonate, hexyltriethyl ammonium carbonate, hepyltriethyl ammonium carbonate, octyltriethyl ammonium carbonate, nonyltriethyl ammonium carbonate, decyltriethyl ammonium carbonate, dodecyltriethyl ammonium carbonate, or undecyltriethyl ammonium carbonate), an amino acid, or a combination thereof.

The amine can be a cyclic amine of 2-(2-chloro-6-fluorophenyl)ethylamine, 1,4-diazabicyclo[2.2.2]octane (DABCO® 33-LV), 1,5-diazabicyclo[4.3.0]non-5-ene, 1,4-diazabicyclo[2.2.2]octane, 1,8-diazabicyclo[5.4.0]undec-7-ene, 4-(dimethylamino)pyridine, 2,6-lutidine, piperidine, 1,8-(dimethylamino)naphthalene, 2,2,6,6-tetramethylpiperidine, 2,8,9-triisobutyl-2,5,8,9-tetraaza-1-phosphabicyclo[3.3.3]undecane, tripelennamine, aniline, benzylamine, N-methyl aniline, imidazole, pyrrole, pyridine, morpholine, or a combination thereof.

The amine can be a hindered secondary amine or a tertiary amine.

The hindered secondary amend can be methyl isopropylamine, ethyl isopropylamine, propyl isopropylamine, butyl isopropylamine, diisopropylamine, dit-butylamine, methyl t-butylamine, ethyl t-butylamine, propyl t-butylamine, methyl secbutylamine, ethyl secbutylamine, propyl secbutylamine, disecbutylamine, methyl isobutylamine, ethyl isobutylamine, proyl isobutylamine, diiosbutylamine, methyl isopentylamine, ethyl isopentylamine, propyl isopentylamine, diisopentylamine, methyl secpentylamine, ethyl secpentylamine, propyl secpentylamine, disecpentylamine, or a combination thereof.

The tertiary amine can be trimethylamine, triethylamine, tripropylamine, tributylamine, dimethylethylamine, dimethylpropylamine, dimethylbutylamine, diethylmethylamine, diethylpropylamine, diethylbutylamine, N,N-diisopropylmethylamine, N-ethyldiisopropylamine, N,N-dimethylethylamine, N,N-diethylbutylamine, 1,2-dimethylpropylamine, N,N-diethylmethylamine, N,N-dimethylisopropylamine, 1,3-dimethylbutylamine, 3,3-dimethylbutylamine, N,N-dimethylbutylamine, or a combination thereof.

The alkanolamine can be 2-amino-2-(hydroxymethyl)-1,3-propanediol (Trizma® base), propanolamine, ethanolamine, diethanolamine, dimethylethanolamine, N-methylethanolamine, triethanolamine, or a combination thereof.

The amino acid can be unsubstituted, N-monosubstituted, or N,N-disubstituted. Non-limiting examples of amino acids include alanine, arginine, asparagine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and derivatives thereof.

The amino acid derivative is preferably a dimethyl glycinate.

Preferably, the liquid comprises an aqueous liquid.

Unless otherwise indicated, the alkyl groups described herein are preferably lower alkyl containing from one to eight carbon atoms in the principal chain and up to 20 carbon atoms. Alkyls can be substituted or unsubstituted and straight or branched chain. Examples of unsubstituted alkyls include methyl, ethyl, n-propyl, i-propyl, n-butyl, i-butyl, s-butyl, t-butyl, n-pentyl, i-pentyl, s-pentyl, t-pentyl, and the like. The term “substituted,” as in “substituted alkyl,” means that various heteroatoms such as oxygen, nitrogen, sulfur, phosphorus, and the like can be attached to the carbon atoms of the alkyl group either in the main chain or as pendant groups. For example, the substituted alkyl groups can have —C—X—C— fragments in the main chain wherein the X is a heteroatom. Further, the substituted alkyl groups can have at least one hydrogen atom bound to a carbon atom replaced with one or more substituent groups such as hydroxy, alkoxy, alkylthio, phosphino, amino, halo, silyl, nitro, esters, ketones, heterocyclics, aryl, and the like.

The abbreviation “PDMS” represents polydimethylsiloxane, the abbreviation “PDMS550” represents polydimethylsiloxane having an average molecular weight of 550 daltons, the abbreviation “TMOS” represents tetramethoxysilane or tetramethyl orthosilicate, the abbreviation “PEG 4600” represents polyethylene glycol having an average molecular weight of 4600 daltons, and the abbreviation “MTMOS” represents methyltrimethoxysilane.

In the examples below the term “carbonate” refers to a 20 wt % potassium carbonate solution (as pure), 30% converted to bicarbonate having a pH of ˜10. The abbreviation “KDMG” represents potassium dimethylglycinate and refers to a 34 wt. % solution of KDMG, 30% converted to bicarbonate having a pH of ˜10.5.

Having described the invention in detail, it will be apparent that modifications and variations are possible without departing from the scope of the invention defined in the appended claims.

### EXAMPLES

The following non-limiting examples are provided to further illustrate the present invention.

### Example 1

### Incorporation of Hydrophobic Additives in the Phosphate Buffer Diluted Synthesis and Entrapment of Carbonic Anhydrase in the Presence of 18-Crown-6 in Polysilicate-Polysiloxane Particles

In a typical procedure, a 250 mL beaker was charged with 1.8 mL of a 150 mg/mL stock solution of carbonic anhydrase (carbonic anhydrase, NS81239, supplied by Novozymes A/S, Denmark).

Next a solution containing tetramethyl orthosilicate (3.3 mL, 21.7 mmol), an alkyl-trimethoxysilane (4.3 mmol) such as isobutyl (0.82 mL) or nonafluorohexyl (1.2 mL) trimethoxysilane, silanol-terminated poly(dimethyl siloxane) (average M_{w}=550; 3.3 mL) and 18-crown-6 ether (0.9 g, 3.3 mmol) was added under vigorous stirring (or vortexing). Upon effective dispersion and subsequent emulsion formation, 45 mL of 50 mM potassium phosphate buffer (pH ˜7.2) containing 150 μL NH₄F catalyst was added to the stirring mixture.

The mixture turned opaque within 15-25 seconds and began to thicken after 45-60 seconds. With longer stirring, the mixture began to thin out enough to be able to transfer it.

It was transferred to a closed container to age for 22 hours at room temperature on a shaker plate.

After aging, the gel product was spray dried.

The spray dried powder was collected from the sample container and cured in a 95° C. oven for 48 hours.

The powder was hydrated in 4 wt. % carbonate (pH ˜10) under stirring/agitation at 45° C. until no dry powder was detected on top of the solution. To facilitate this initial hydration/washing process, acetone or ethanol (5-25 vol %) can be added. The particles were then collected via filtration and dried at 55° C. overnight, and an aliquot of the hydration solution was collected to determine enzyme retention in the particles.

Alternatively, before drying, the particles were placed in an acetone/water mixture at room temperature under stirring/agitation for 24-48 hours and then collected via filtration.

The particles were the placed in pure acetone at room temperature under stirring/agitation for 24-48 hours. They were then collected via filtration and dried at 55° C. overnight to remove any residual organic solvent.

These dried, washed particles were then rehydrated in the solvent of choice (potassium dimethyl glycine (KDMG) or carbonate) at 45° C. in shaker/incubator for 2 days before testing for activity.

To characterize the buoyancy of the particles, a method was developed to monitor the rate separation of the particles from the solution (by floatation) using optical transmission. The method includes placing a well-mixed aliquot of the material, suspended in potassium dimethyl glycine (KDMG), into a 3 mL cuvette. The cuvette containing the sample is incubated in a temperature-controlled sample cell until it reaches the desired temperature. The sample is briefly removed, inverted several times to ensure adequate mixing, and immediately placed into a UV/Vis spectrometer. The spectrometer is setup to monitor light absorption/transmission at 450 nm over time. At the beginning of the run the biocatalyst materials obscure the light from reaching the detector. As the particles float to the top of the cuvette the solution clarifies allowing more light to pass through. Slowly separating particles yield flat profiles while quickly separating particles yield dynamic curves.

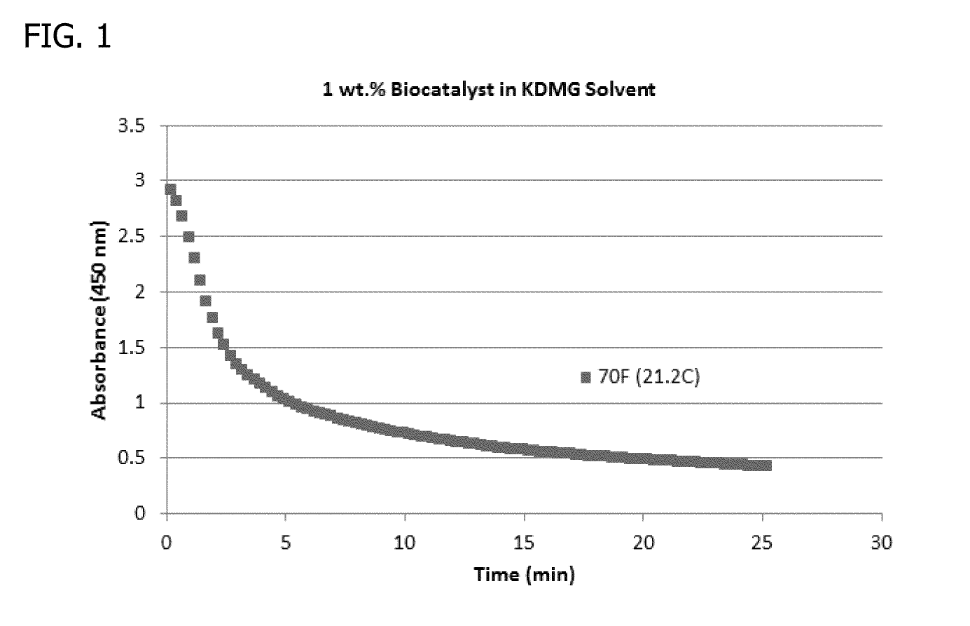

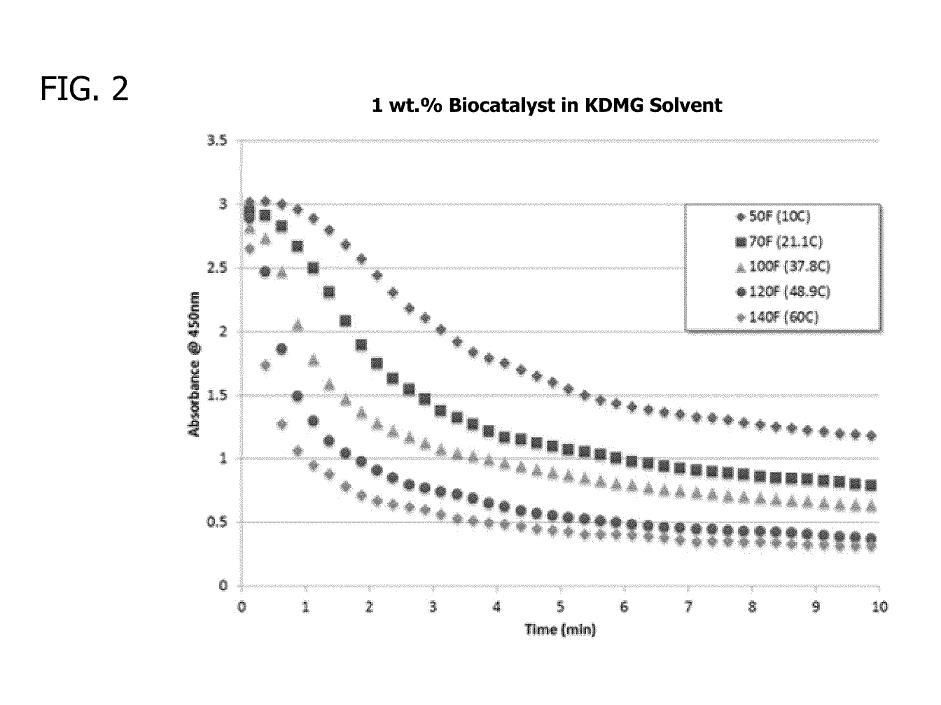

FIG. 1 depicts the separation of the biocatalyst material over 30 minutes at 70° F. FIG. 2 shows the effect of temperature on biocatalyst buoyancy. There is a direct correlation between temperature and the rate of particle separation in KDMG. The biocatalyst at 120° F. and 140° F. reach the same end point as the 70° F. material in about one-third the time. It is believed that increased rate of separation is due to a reduction in the solution viscosity as the viscosity of KDMG at RT (20° C.) is 4.0 cP, but drops to 1.6 cP at 60° C.

Another buoancy experiment used light absorption to track the clarification process. The same sample presented in FIG. 2 demonstrated that a progressive clarification (decreased absorbance with time) was achieved within a few minutes, and this clarification process was enhanced with elevated solution temperatures.

### Example 2

### Neat Synthesis and Entrapment of Carbonic Anhydrase in the Presence of 18-Crown-6 in Polysilicate-Polysilicone Particles

In a typical procedure, a 100 mL beaker was charged with tetramethyl orthosilicate (2.6 mL, 17.6 mmol), silanol-terminated poly(dimethyl siloxane) (average M_{w}=550; 2.2 mL) and 18-crown-6 ether (0.6 g, 2.2 mmol). The mixture was stirred vigorously for 5 minutes to fully dissolve the 18-crown-6 ether.

Separately, a 150 mg/mL stock solution of carbonic anhydrase (carbonic anhydrase, NS81239, supplied by Novozymes A/S, Denmark) was prepared in 10 mM phosphate buffer (pH=7.2).

Next, 2.4 mL of the CA stock solution was added to the reaction beaker and the resulting heterogeneous mixture was stirred vigorously to generate a finely dispersed emulsion.

Next 112 μL of a 1 M aqueous solution NH₄F were added to the reaction vessel. The mixture was subsequently stirred for 2 minutes, or until gelation began to be observed, via magnetic stirring. After 2 minutes of stirring, the beaker contained a highly viscous polymerized material that could no longer be mixed through stirring.

The polymerized material was then dried at room temperature for 1 hour. After 1 hour, the beaker was transferred to a 55° C. oven for 24 hours, after which the temperature was increased to 75° C. for 72 hours.

After curing, the powder was collected, ground using a mortar and pestle, and then the dry mass was obtained.

In some cases, after obtaining the dry mass, the bulk powder was separated by size using a series of sieves.

The powder with the desired particle size was hydrated in either carbonate or KDMG for 72 hours under stirring and/or agitation. The particles were then collected via filtration, and an aliquot of the storage solution was collected to determine enzyme retention in the particles.

These particles were then washed thoroughly with fresh solution and analyzed for activity.

### Example 3

### Phosphate Buffer Diluted Synthesis and Entrapment of Carbonic Anhydrase in the Presence of 18-Crown-6 in Polysilicate-Polysilicone Particles

In a typical procedure, a 100 mL beaker was charged with 2.4 mL of a 150 mg/mL stock solution of carbonic anhydrase (carbonic anhydrase, NS81239, supplied by Novozymes A/S, Denmark). Next, 5.0 mL of 100 mM phosphate buffer was added and mixed thoroughly.

Next a solution containing tetramethyl orthosilicate (2.6 mL, 17.6 mmol), silanol-terminated poly(dimethyl siloxane) (average M_{w}=550; 2.2 mL) and 18-crown-6 ether (0.6 g, 2.2 mmol) was added under vigorous stirring (or vortexing).

A volume of 112 μL of a 1 M aqueous solution NH₄F was added to the reaction vessel. The mixture was subsequently stirred until gelation began to be observed.

The sample was then cured and hydrated following the procedure described in Example 2.

### Example 4

### Methanol Diluted Synthesis and Entrapment of Carbonic Anhydrase in the Presence of 18-Crown-6 in Polysilicate-Polysilicone Particles

In a typical procedure, a 400 mL beaker was charged with 12 mL of a 150 mg/mL stock solution of carbonic anhydrase (carbonic anhydrase, NS81239, supplied by Novozymes A/S, Denmark). Next a solution containing tetramethyl orthosilicate (13 mL, 88 mmol), silanol-terminated poly(dimethyl siloxane) (average M_{w}=550; 11 mL) and 18-crown-6 ether (3 g, 11 mmol) was added under vigorous stirring (or vortexing).

Upon effective dispersion and subsequent emulsion formation, 15 mL of reagent grade methanol were added. Immediately after addition of the methanol, 0.5 mL of 1 M NH₄F was added to the stirring mixture. The mixture began to thicken after 30 seconds, and gelation was observed in approximately 30 minutes.

The same procedure for thermal curing, hydration, and detection of enzyme retention that was described in Example 2 was used here.

### Example 5

### Methanol Diluted Synthesis and Entrapment of Carbonic Anhydrase in the Presence of 18-Crown-6 in Polysilicate-Polysilicone Particles; Alternate Order of Addition

In a typical procedure, a 400 mL beaker was charged with 12 mL of a 150 mg/mL stock solution of carbonic anhydrase (carbonic anhydrase, NS81239, supplied by Novozymes A/S, Denmark). Next, 0.5 mL of 1 M NH₄F was added to the enzyme solution under stirring.

A separate stock solution of organic monomers (Stock B) was prepared by combining tetramethyl orthosilicate (13 mL, 88 mmol), silanol-terminated poly(dimethyl siloxane) (average M_{w}=550; 11 mL), and 25 mL of methanol.

Separately, a stock solution of 18-crown-6 (3 g, 11 mmol; Stock C) and 25 mL of reverse osmosis (RO) water was prepared.

Stock solutions B and C were then combined and added to the beaker containing enzyme under vigorous stirring. The mixture began to thicken quickly, and gelation was observed in approximately 30 seconds.

The same procedure for thermal curing, hydration, and detection of enzyme retention that was described in Example 2 was used here.

### Example 6

### Methanol Diluted Synthesis and Entrapment of Carbonic Anhydrase in the Presence of Poly(Ethylene Glycol) in Polysilicate-Polysilicone Particles

Similar to Example 5, a 400 mL beaker was charged with 12 mL of a 150 mg/mL stock solution of carbonic anhydrase (carbonic anhydrase, NS81239, supplied by Novozymes A/S, Denmark). Next, 0.5 mL of 1 M NH₄F was added to the enzyme solution under stirring. A separate stock solution of organic monomers (Stock B) was prepared by combining tetramethyl orthosilicate (13 mL, 88 mmol), silanol-terminated poly(dimethyl siloxane) (average M_{w}=550; 11 mL), and 25 mL of methanol.

Separately, a stock solution of polyethylene glycol, Mᵣ=570-630) (3 g; Stock C) and 25 mL of RO water was prepared.

Stock solutions B and C were then combined and added to the beaker containing enzyme under vigorous stirring. The mixture began to thicken quickly, and gelation was observed in approximately 30 seconds.

The same procedure for thermal curing, hydration, and detection of enzyme retention that was described in Example 2 was used here.

This procedure was also used to make derivative containing polyethylene glycol (typical Mₙ=4,600), polyethylene glycol (typical Mₙ=8,000), and poly(ethylene oxide) (approximate M_{w}=100,000).

### Example 7

### Incorporation of Hydrophobic Additives in the Methanol Diluted Synthesis and Entrapment of Carbonic Anhydrase in the Presence of 18-Crown-6 in Polysilicate-Polysilicone Particles

Similar to Example 4, a 400 mL beaker was charged with 12 mL of a 150 mg/mL stock solution of carbonic anhydrase (carbonic anhydrase, NS81239, supplied by Novozymes A/S, Denmark). Next, a solution containing tetramethyl orthosilicate (11.8 mL, 80 mmol), trimethoxymethyl silane (1.14 mL; 8 mmol), silanol-terminated poly(dimethyl siloxane) (average M_{w}=550; 11 mL) and 18-crown-6 ether (3 g, 11 mmol) under vigorous stirring (or vortexing).

Upon effective dispersion, subsequent emulsion formation, 15 mL of reagent grade methanol were added. Immediately after addition of the methanol, 0.5 mL of 1 M NH₄F was added to the stirring mixture.

Gelation was observed in approximately 30 minutes.

The same procedure for thermal curing, hydration, and detection of enzyme retention that was described in Example 2 was used here.

This procedure was also used to prepare xerogels containing derivatives containing iso-butyl(trimethoxy)silane, n-butyl(trimethoxy)silane, and n-octyl(trimethoxy)silane in a 1:10 molar ratio with tetramethylorthosilicate.

### Example 8

### Ethanol Diluted Synthesis and Entrapment of Carbonic Anhydrase in the Presence of 18-Crown-6 and CTAB in Polysilicate-Polysilicone Particles

In a typical procedure, a 250 mL beaker was charged with 3.6 mL of a 150 mg/mL stock solution of carbonic anhydrase (carbonic anhydrase, NS81239, supplied by Novozymes A/S, Denmark). To this enzyme solution, 156 μL of a 160 mg/mL solution of cetyl trimethylammonium bromide (CTAB, 0.067 mmol) dissolved in 95% water, 5% ethanol was added and stirred to homogenize.

Next a solution containing tetramethyl orthosilicate (3.9 mL, 26 mmol), silanol-terminated poly(dimethyl siloxane) (average M_{w}=550; 3.3 mL) and 18-crown-6 ether (0.9 g, 3.3 mmol) was added under vigorous stirring (or vortexing). Upon effective dispersion and subsequent emulsion formation, 4.5 mL of 0.1 M NH₄F was added to the stirring mixture.

The mixture began to turn opaque immediately, and 27 mL of reagent alcohol (90% ethanol, 5% methanol, 5% isopropanol) was quickly added under vigorous stirring.

After 10-15 seconds, this mixture began to thicken, and a second aliquot of 7.5 mL of reagent alcohol was added with stirring to facilitate transfer of the mixture.

It was then poured into a shallow container (6″×6″×1″) and left to dry overnight at room temperature.

The following day, the container was placed in a 75° C. oven for 72 hours.

The same procedure for powder processing, hydration, and detection of enzyme retention that was described in Example 2 was used here.

### Example 9

### Phosphate Buffer Diluted Synthesis and Entrapment of Carbonic Anhydrase in the Presence of 18-Crown-6 and CTAB in Polysilicate-Polysilicone Particles

In a typical procedure, a 250 mL beaker was charged with 3.6 mL of a 150 mg/mL stock solution of carbonic anhydrase (carbonic anhydrase, NS81239, supplied by Novozymes A/S, Denmark). To this enzyme solution, 156 μL of a 160 mg/mL solution of cetyl trimethylammonium bromide (CTAB, 0.067 mmol) dissolved in 95% water, 5% ethanol was added and stirred to homogenize.

Next a solution containing tetramethyl orthosilicate (3.9 mL, 26 mmol), silanol-terminated poly(dimethyl siloxane) (average M_{w}=550; 3.3 mL) and 18-crown-6 ether (0.9 g, 3.3 mmol) was added under vigorous stirring (or vortexing). Upon effective dispersion and subsequent emulsion formation, 45 mL of 100 mM potassium phosphate buffer (pH ˜7.2) containing 300 μL NH₄F of 3 M catalyst was added to the stirring mixture.

The mixture turned opaque within 7-10 seconds and began to thicken after 25-30 seconds. With longer stirring, the mixture began to thin out enough to be able to transfer it.

It was then poured into a shallow container (6″×6″×1″) and left to dry overnight at room temperature.

The following day, the container was placed in a 75° C. oven for 72 hours.

The same procedure for powder processing, hydration, and detection of enzyme retention that was described in Example 2 was used here.

Alternatively, the gel product obtained after the addition of phosphate buffer and NH₄F catalyst was spray dried.

The spray dried powder was collected from the sample container and cured in a 85° C. oven for 72 hours.

The same procedure for hydration and detection of enzyme retention that was described in Example 2 was used for the spray dried powder as well.

### Example 10

### Hydrophobic Post-Modification of Polysilicate-Polysilicone Particles Containing Carbonic Anhydrase

In a typical procedure, a 500 mL round bottom flask was charged with 200 mL of dry toluene, 2.0 g of xerogel particles derived from Example 8, and a magnetic stir bar.

The flask was purged with nitrogen to remove moisture from the head space, and 0.25 mL of diethylenetriamine (an acid scavenger) was added via syringe.

Nonafluorohexyldimethylchlorosilane (1.26 mL, 5 mmol) was then added via syringe, and the suspension was stirred overnight at room temperature.

The surface modified xerogel particles were then collected via gravity filtration and washed with toluene, ethanol, water, and finally ethanol.

The particles were then dried in a vacuum oven at 60° C. for 3 hours.

The same procedure for hydration and detection of enzyme retention that was described in Example 2 was used here.

This procedure was also used to make xerogel particles modified with benzyldimethylchlorosilane, octyldimethylchlorosilane, and octadecyldimethylchlorosilane.

### Example 11

### Characterization of Polysilicate-Polysilicone Particles Containing Carbonic Anhydrase

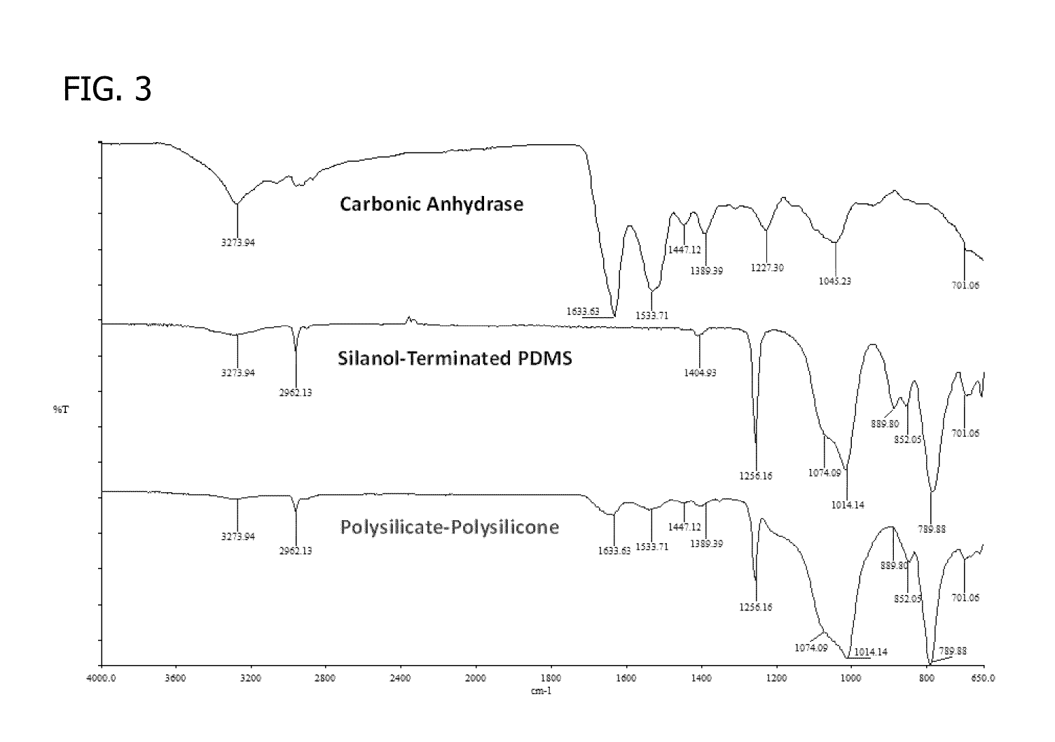

A representative FTIR spectrum of polysilicate-polysilicone particles containing encapsulated carbonic anhydrase is shown in FIG. 3 as compared to PDMS only and carbonic anhydrase only. Diagnostic peaks corresponding to Si—O bonding are seen at 794 cm⁻¹ and 1013 cm⁻¹. Further, the peaks at 1257 cm⁻¹ and 2960 cm⁻¹ indicate successful incorporation of the PDMS. The immobilized enzyme shows a series of peaks from 1375 cm⁻¹ to 1680 cm⁻¹. These spectral features are typically found in xerogel particles prepared using the combination of tetramethylorthosilicate, polydimethylsiloxane, and carbonic anhydrase in the ratios described in Examples 2 through 9.

Typically, the incorporation of 18-crown-6 and polyethylene glycol is not identifiable by FTIR analysis as their main diagnostic peaks are overlapped by the other components of the matrix.

A series of representative Brunner-Emmett-Teller (BET) nitrogen sorption isotherms were collected on samples prepared according to the experimental procedures described in Example 4 and Example 8. The average surface area (m²/g) ranged from 5.83 to 18.42 m²/g, with an average of surface area of 10.4 m²/g.

The pore volume distributions obtained from Barrett-Joyner-Halenda (BJH) analysis on the previous samples showed pore volumes (cc/g) ranging from 0.0344 to 0.172, with calculated average pore volume of 0.059 cc/g.

The pore diameters obtained from aforementioned BJH analysis showed median pore sizes ranged from 10 nm to 80 nm.

The sample prepared according to the methods in Example 8 showed the highest surface area (18.42 m²/g) and pore volume (0.172 cc/g) in this series.

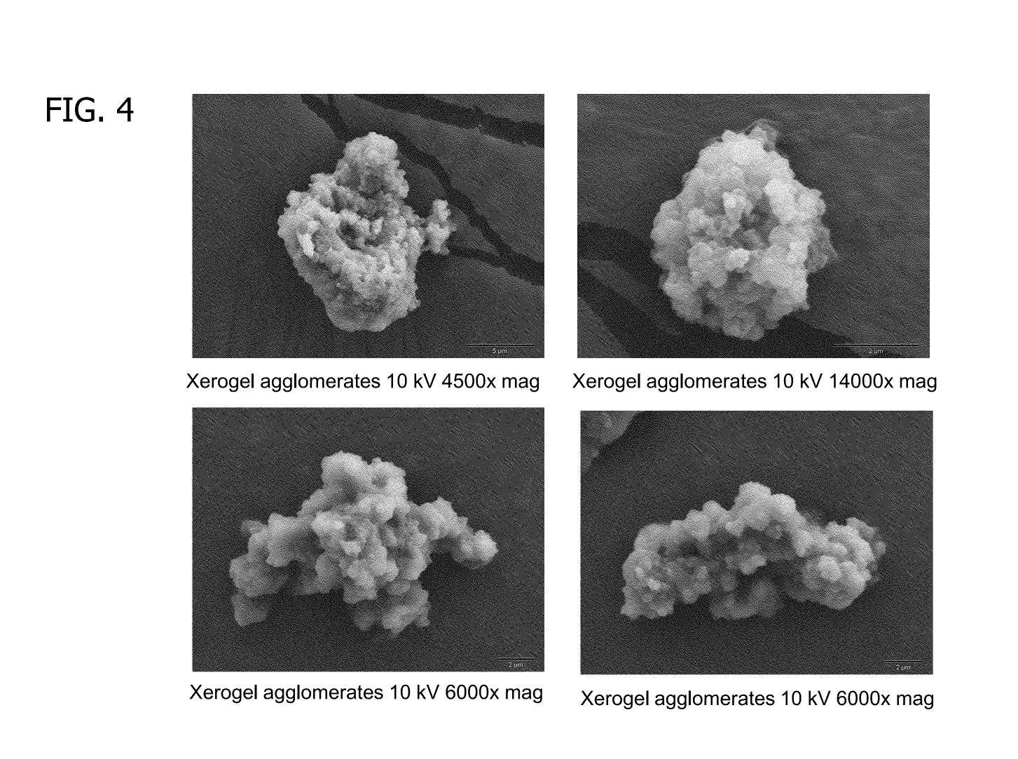

A series of scanning electron microscopy images (SEM) of representative polysilicate-polysilicone particles containing encapsulated carbonic anhydrase are shown in FIG. 4. The representative images show particle agglomerates that are roughly 10 to 20 μm across. The agglomerates appear to be composed of smaller primary particles.

### Example 12

### Activity Testing of Polysilicate-Polysilicone Particles Containing Carbonic Anhydrase in a Batch Reactor

To test the activity of polysiloxane-polysilicate particles containing carbonic anhydrase, suspensions were prepared at different weight percentages of particles and tested in a batch reactor system.

The batch reactor consisted of a sealed vessel that is pressurized between 60 and 100 psig. The feed gas consisted of 15% CO₂ balanced with N₂. The experiments described herein were conducted at room temperature.

The gas phase was mixed using a mechanical stirring rod, and the liquid phase was mixed with a magnetic stir bar. The mixing of the liquid was slow enough that the surface area remains unchanged throughout the experiment.

After charging the vessel with CO₂ mixture the pressure drop in the vessel was monitored over a 10 minute time period. Using the known number of moles of CO₂ in the vessel, the pressure, and the surface area of the solution, the mass transfer coefficient (K_{G}) of the suspensions was calculated and expressed in mmol/s·m²·kPa. This mass transfer coefficient was divided by the value for blank solvent under the same conditions to get the rate multiplier shown in Tables 1 and 2 below.

Typically, samples were tested in 100 mL of either a carbonate or KDMG solution with varying weight percent loadings and/or the amount of enzyme present in the reactor.

Table 1 shows the activity of some representative samples prepared according to the methods described in Examples 2 through 9. The samples shown in Table 1 represent a wide range of synthesis methods, formulations, weight percent loadings, and processing.

TABLE 1
Activities of polysilicate-polysiloxane particles
in a batch reactor in carbonate solution

| | | Weight | | |
|---|---|---|---|---|
| | | Percent of | Xerogel | |
| | Sample | xerogel in | Processing | |
| Entry | preparation | suspension | Method | Rate multiplier |
| 1 | Example 4; No | 0.20% | #1 | 1.1 |
| | enzyme | | (see | |
| | | | description | |
| | | | below) | |
| 2 | Example 8 | 0.20% | #1 | 6.8 |
| 3 | Example 4 | 0.20% | #1 | 7.0 |
| 4 | Example 5 | 0.20% | #3 | 5.7 |
| 5 | Example 6 | 0.30% | #3 | 7.8 |
| 6 | Example 6; | 0.20% | #3 | 10.7 |
| | PEG₄₆₀₀ | | | |
| 7 | Example 6; | 0.30% | #3 | 9.3 |
| | PEG₈₀₀₀ | | | |
| 8 | Example 7 | 0.20% | #3 | 10.8 |
| 9 | Example 7; | 0.20% | #3 | 8.5 |
| | TMOS/n-butyl | | | |
| | TMOS | | | |
| 10 | Example 7; | 0.20% | #3 | 12.1 |
| | TMOS/iso-butyl | | | |
| | TMOS | | | |
| 11 | Example 3 | 0.20% | #3 | 14.3 |
| 12 | Example 3; No | 0.20% | #3 | 1.9 |
| | PDMS | | | |
| 13 | Example 3; No | 0.20% | #3 | 12.2 |
| | 18-crown-6 | | | |
| 14 | Example 9 | 0.20% | #1 | 8.5 |
| 15 | Example 9 | 0.20% | #2 | 9.9 |

Xerogel processing method #1 comprises milling and sieving particles to <125 μm and hydrating and dispersing via mechanical stirring. Method #2 comprises spray drying to produce particles and then hydrating and dispersing via mechanical stirring. Method #3 comprises hydrating via mechanical stirring and then sonication to disperse the particles.

Entry #1 in Table 1 was a representative negative control, prepared by the synthetic procedure described in Example 4, but with phosphate buffer used to replace the carbonic anhydrase solution. The rate multiplier was 1.1, indicating that this sample had comparable activity to blank solvent. This result clearly indicates that xerogel particles without carbonic anhydrase do not accelerate CO₂ absorption into the test solution.

Entries #2-15 in Table 1 describe the activities of sample prepared in the presence of carbonic anhydrase according to the methods described in Examples 2 to 9. With the exception of entry #12, all samples showed considerable enhancement over the blank solvent.

Entry #12 in Table 1 shows a sample prepared according to Example 3 in the absence of silanol terminated PDMS₅₅₀. After washing and hydration, quantitation of the enzyme suggested little to no enzyme was encapsulated in the remaining polysilicate particles. This was evident in the very low activity at 0.2 wt. % xerogel loading.

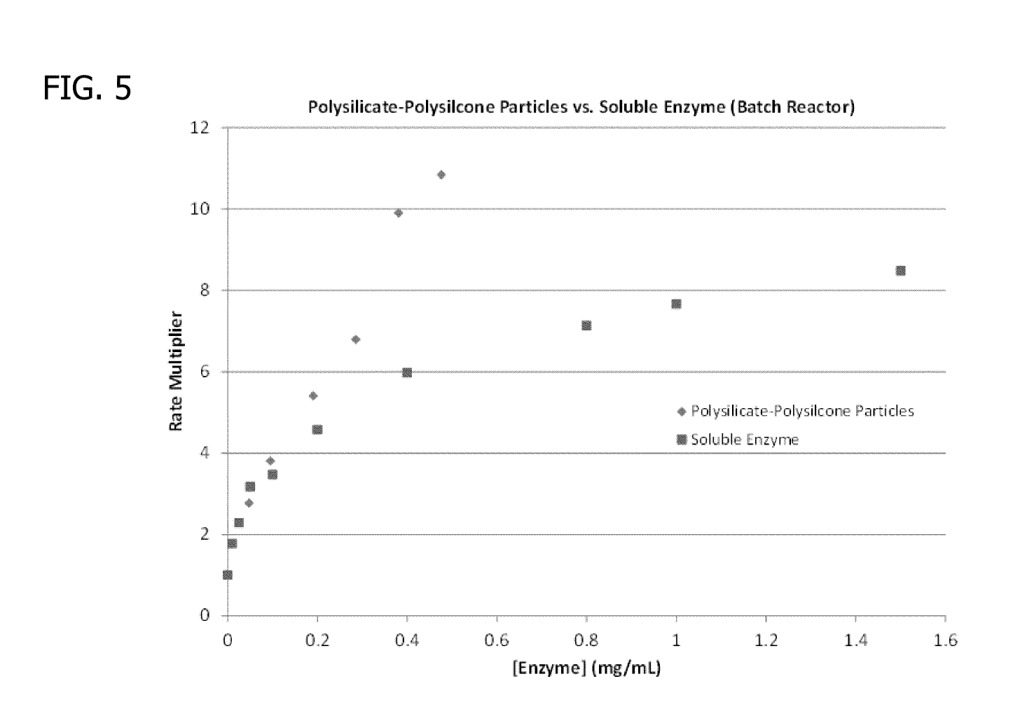

In FIG. 5, a representative loading study of a sample prepared according to the synthetic methods described in Example 8 is shown compared to soluble enzyme. The polysilicate-polysilicone particles have comparable activity to soluble enzyme at low enzyme loadings. At higher enzyme loadings, the activity of the polysilicate-polysilicone particles is higher than soluble enzyme. This result results from the concentration of the particles at the gas-liquid interface of the batch reactor due to their low density.

Table 2 shows the activity of some representative samples prepared according to the methods described in Example 10. The entries in Table 2 show the activity of several hydrophobically modified xerogel particles in both carbonate and KDMG solutions.

TABLE 2
Activities of hydrophobically modified
polysilicate-polysiloxane particles in a batch reactor

| | | Weight | | |
|---|---|---|---|---|
| | | Percent of | | |
| | | xerogel in | | |
| | Hydrophobic | suspension | | Rate |
| Entry | Surface Modifier | (%) | Solvent | Multiplier |
| 1 | None | 0.20% | Carbonate | 6.5 |
| 2 | Benzyldimethylchlorosilane | 0.20% | Carbonate | 7.4 |
| 3 | Nonafluorohexyldimethyl- | 0.20% | Carbonate | 12.4 |
| | chlorosilane | | | |
| 4 | Octyldimethylchlorosilane | 0.20% | Carbonate | 6.6 |
| 5 | Octadecyldimethyl- | 0.20% | Carbonate | 7.9 |
| | chlorosilane | | | |
| 6 | None | 0.20% | KDMG | 6.2 |
| 7 | Benzyldimethylchlorosilane | 0.20% | KDMG | 11.2 |
| 8 | Nonafluorohexyldimethyl- | 0.20% | KDMG | 3.7 |
| | chlorosilane | | | |
| 9 | Octyldimethylchlorosilane | 0.20% | KDMG | 8.3 |
| 10 | Octadecyldimethyl- | 0.20% | KDMG | 6.6 |
| | chlorosilane | | | |

Entries #2-5 show the effect of various types of hydrophobic post-modification of xerogel particles in a carbonate solution relative to unmodified particles (entry #1). The best improvement in activity (e.g., K_{G}) was seen using the fluoroalkyl modifier (entry #3).

Entries #7-10 show the effect of those same hydrophobically modified xerogel particles in a KDMG solution relative to unmodified particles (entry #6). The best improvement in activity was seen using the benzyl modifier (entry #7).

In contrast to what was observed in carbonate solution, the fluoroalkyl modified xerogel particles in KDMG solution (entry #8) were significantly lower in activity relative to the unmodified particles. This result indicates that the relative hydrophobicity/hydrophilicity of the xerogel particles can be tuned to optimize the performance in the solvent of choice.

### Example 13

### Activity Testing of Polysilicate-Polysiloxane Particles Containing Carbonic Anhydrase in a Continuous Flow Reactor Using Random Packing

To evaluate the use of these particles in a flow-through reactor, a suspension of particles was pumped over random packing (Tipton ⅛ in. ceramics).

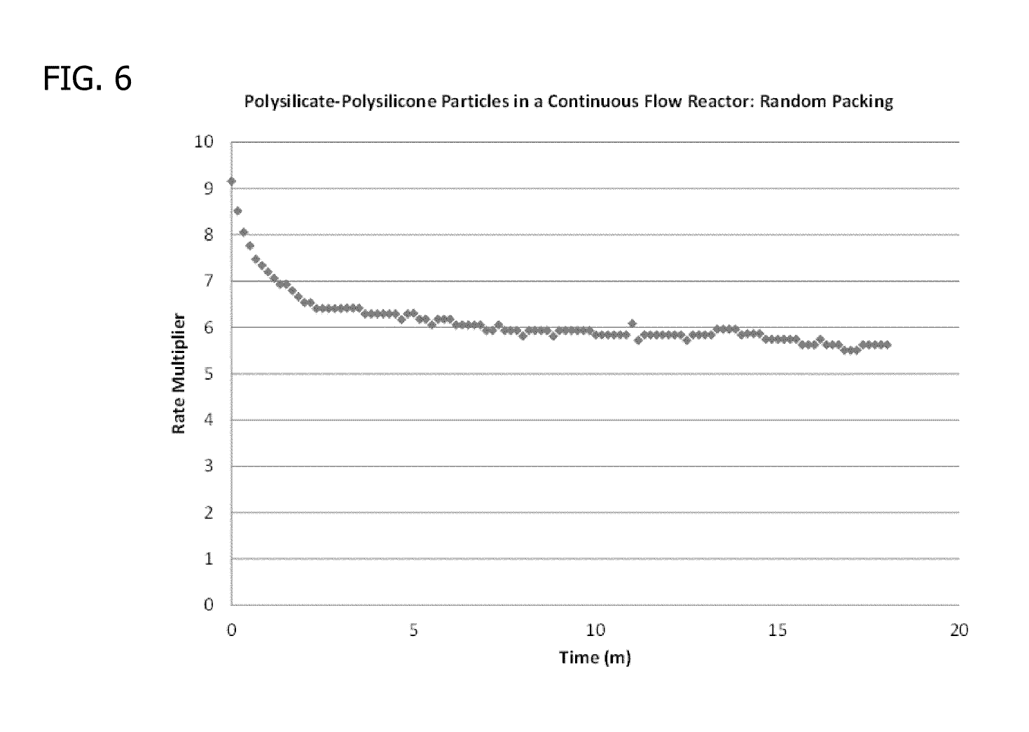

In FIG. 6, a 1.2 wt. % suspension of particles in carbonate solution prepared according to Example 2 was pumped using a peristaltic pump over 65 g of ⅛ in. Tipton ceramic spheres at a controlled rate (20 mL/minutes) from the top of the column. A gas comprising 15% CO₂ (balanced with N₂) was flowed upwards from the bottom of the column. Quantitation of CO₂ conversion was performed using a non-dispersive infrared detector (NDIR) monitoring the CO₂ gas at the output of the column. The differential between the CO₂ content of the output gas versus the feed gas was used to calculate the rate of absorption.

The study in FIG. 6 was conducted for approximately 17 minutes. The sample showed a steady state rate multiplier of approximately 6.0.

### Example 14

### Activity Testing of Polysilicate-Polysiloxane Particles Containing Carbonic Anhydrase in a Continuous Flow Reactor Using Structured Packing

Various xerogel suspensions were used in flow-through studies over Sulzer Mellapak® 500X structured packing with a 2″ column diameter. The packing surface was modified using a two-part epoxy and silica gel to create a hydrophilic/textured surface that spreads out the liquid and improves the gas-liquid contact area. All xerogel suspensions were pumped at a controlled rate (218 mL/minute) from the top of the column. A gas comprising 15% CO₂ (balanced with N₂) was flowed upwards from the bottom of the column (2.18 SLPM). Quantitation of CO₂ conversion was performed using a non-dispersive infrared detector (NDIR) monitoring the CO₂ gas at the output of the column. The differential between the CO₂ content of the output gas versus the feed gas was used to calculate the rate of absorption.

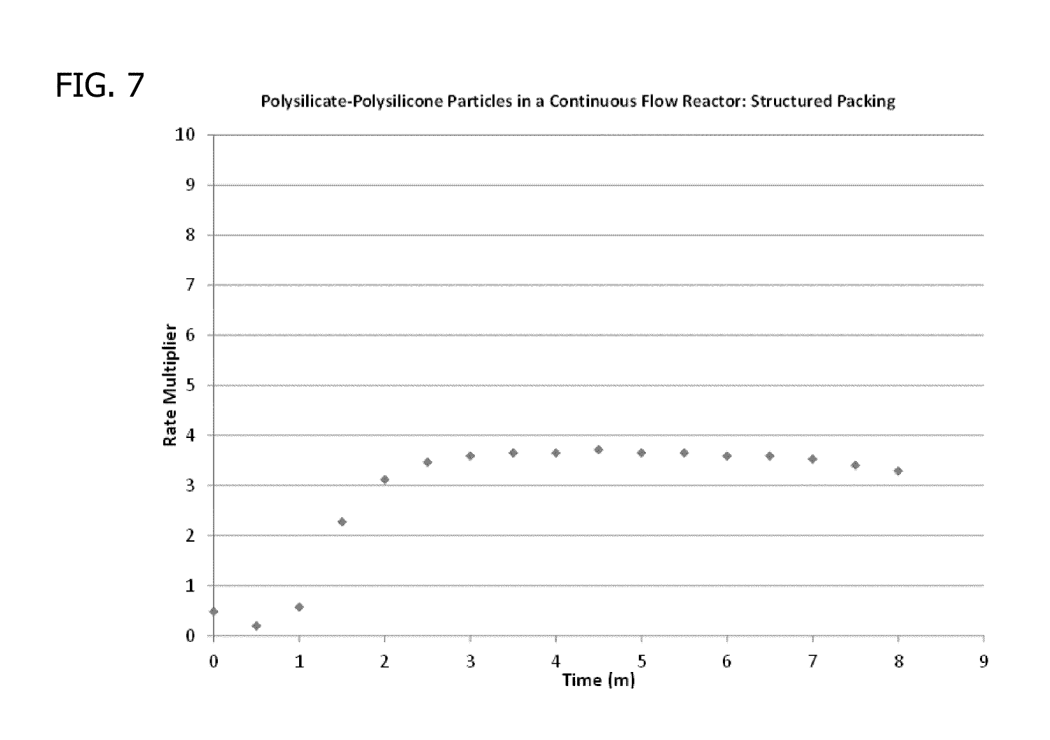

In FIG. 7, a 0.5 wt % suspension of particles in carbonate solution prepared according to Example 9 was pumped using a peristaltic pump over a single unit of structured packing. This sample showed a steady state rate multiplier of around 3.5.

Table 3 shows the activity of some representative samples prepared according to the methods described in Examples 8 through 10. The samples shown in Table 3 represent a range of synthesis methods, formulations, and weight percent loadings in both carbonate and KDMG solutions.

| | | Weight | | | |
|---|---|---|---|---|---|
| | | Percent of | Xerogel | | |
| | | xerogel in | Processing | | Rate |
| Entry | Sample preparation | suspension | Method | Solvent | Multiplier |
| 1 | Example 8 | 0.20% | #1 | Carbonate | 2.1 |
| | | | (see | | |
| | | | description | | |
| | | | below) | | |
| 2 | Example 8 | 0.50% | #1 | Carbonate | 2.1 |
| 3 | Example 8 | 1.0% | #1 | Carbonate | 2.3 |
| 4 | Example 10 | 0.50% | #1 | Carbonate | 3.6 |
| 5 | Example 9 | 0.20% | #1 | Carbonate | 2.3 |
| 6 | Example 9 | 0.50% | #1 | Carbonate | 3.4 |
| 7 | Example 9 | 1.5% | #1 | Carbonate | 4.0 |
| 8 | Example 9 | 2.4% | #1 | Carbonate | 3.6 |
| 9 | Example 10 using | 0.50% | #1 | Carbonate | 3.7 |
| | particles generated as | | | | |
| | described in example 8 | | | | |
| 10 | Example 9 but cured at | 0.50% | #2 | Carbonate | 3.4 |
| | 75° C. | | | | |
| 11 | Example 9 | 0.50% | #2 | Carbonate | 3.6 |
| 12 | Example 9 except that | 0.50% | #2 | Carbonate | 3.6 |
| | 50 mM phosphate and | | | | |
| | 150 μL of NH₄F was | | | | |
| | used | | | | |
| 13 | Example 9 except that | 0.50% | #2 | Carbonate | 4.1 |
| | 50 mM phosphate and | | | | |
| | 150 μL of NH₄F was | | | | |
| | used cured 72 hours at | | | | |
| | 95° C. | | | | |
| 14 | Example 8 | 0.20% | #1 | KDMG | 1.3 |
| 15 | Example 8 | 0.50% | #1 | KDMG | 1.4 |
| 16 | Example 8 | 1.0% | #1 | KDMG | 2.0 |
| 17 | Example 8 | 2.0% | #1 | KDMG | 2.2 |
| 18 | Example 9 except that | 1.0% | #1 | KDMG | 1.6 |
| | 50 mM phosphate and | | | | |
| | 150 μL of NH₄F was | | | | |
| | used | | | | |
| 19 | Example 9 except that 50 mM | 1.0% | #2 | KDMG | 2.5 |
| | phosphate and 150 μL | | | | |
| | of NH₄F was used | | | | |

Xerogel processing method #1 comprises milling and sieving particles to <125 μm and hydrating and dispersing via mechanical stirring. Method #2 comprises spray drying to produce particles and then hydrating and dispersing via mechanical stirring.

Entries #1-3 show a weight loading study using ethanol-diluted xerogel particles derived from Example 8 in carbonate. This study shows that there is not an appreciable activity dependence on loading using this type of xerogel particle.

In contrast, entries #5-8 show a weight loading study using phosphate-diluted particles derived from Example 9 in carbonate, clearly showing an activity dependence on wt loading up to 1.5%. These particles also show better activity than those derived from Example 8 at comparable loadings (entry #6 vs. #2).

The activity of the ethanol-diluted xerogels in carbonate can be significantly improved through hydrophobic post-modification of their surface using a fluoroalkyl modifier (entry #4 vs. #2 for nonmodified particles).

Hydrophobic post-modification using a fluoroalkyl modifier of the phosphate-diluted particles derived from Example 9 also shows improved activity in carbonate at the same wt % (entry #9 vs. #6 for nonmodified particles), albeit less significant of an improvement relative to the ethanol-diluted particles.

Phosphate-diluted xerogel particles generated via Example 9 using a spray dryer have at least comparable (entry #10) to better (entries #11-13) activities in carbonate than those generated using the same formulation but mechanically ground and sieved to <125 μm (entry #6).

Entries #14-17 show a weight loading study using ethanol-diluted xerogel particles derived from Example 8 in KDMG. In contrast to carbonate, there is a clear activity dependence on loading using these particles.

At comparable loadings, the activity of these particles is lower in KDMG (entry #14) than in carbonate (entry #1). It has been observed that xerogel particles disperse more homogeneously in KDMG than in carbonate. Better dispersion results in these particles existing farther from the gas-liquid interface, increasing the average diffusion path length for CO₂ to reach the enzyme and reducing the observed activity.

In KDMG, phosphate-diluted xerogel particles generated via Example 9 using a spray dryer have significantly better activity (entry #19) than those generated using the same formulation but mechanically ground and sieved to <125 μm (entry #18).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there can be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

1. A carbonic anhydrase xerogel particle comprising carbonic anhydrase entrapped within a xerogel particle, the carbonic anhydrase xerogel particle being derived from a sol comprising (i) an alkoxy silane, an organotrialkoxy silane, or a metasilicate, (ii) a hydrophobic organotrialkoxy silane, a hydrophobic organohalosilane, or a combination thereof, (iii) a poly(silicone), (iv) a hydrophilic additive, and (v) carbonic anhydrase.

2. The carbonic anhydrase xerogel particle of claim 1 wherein the hydrophobic organotrialkoxy silane or hydrophobic organohalosilane comprises an alkyl chlorosilane, a fluoroalkyl chlorosilane, a phenyl chlorosilane, an alkyl trialkoxysilane, a fluoroalkyl trialkoxysilane, a phenyl trialkoxysilane, or a combination thereof.

3. The carbonic anhydrase xerogel particle of claim 2 wherein the hydrophobic organotrialkoxy silane or hydrophobic organohalosilane comprises octadecyldimethylchlorosilane, benzyldimethyl chlorosilane, octadecyl trimethoxysilane, phenylethyl trimethoxysilane, nonafluorohexyl trimethoxysilane, (3,3,3-trifluoropropyl)dimethyl chlorosilane, isobutyl trimethoxysilane, nonafluorohexyldimethyl chlorosilane, tridecafluoro-1,1,2,2-tetrahydrooctyl)dimethyl chlorosilane, (heptadecafluoro-1,1,2,2-tatrahydrodecyl)dimethyl chlorosilane, or a combination thereof.

4. The carbonic anhydrase xerogel particle of claim 3 wherein the hydrophobic organotrialkoxy silane comprises nonafluorohexyl trimethoxysilane, isobutyl trimethoxysilane, or a combination thereof.

5. The carbonic anhydrase xerogel particle of claim 1 wherein the alkoxy silane is tetramethylorthosilicate, tetraethylorthosilicate, methyltriethylorthosilicate, ethyltrimethylorthosilicate, dimethyldiethylorthosilicate, tetraglyceryl silicate, or a combination thereof.

6. The carbonic anhydrase xerogel particle of claim 5 wherein the organotrialkoxy silane is trimethoxymethylsilane, trimethoxyethylsilane, or a combination thereof.

7. The carbonic anhydrase xerogel particle of claim 1 wherein the alkoxy silane comprises tetramethylorthosilicate.

8. The carbonic anhydrase xerogel particle of claim 1 wherein the poly(silicone) is a poly(siloxane) selected from the group consisting of poly(dimethylsiloxane), poly(dimethylsiloxane)-co-poly(alkene oxide), or a combination thereof.

9. The carbonic anhydrase xerogel particle of claim 8 wherein the poly(siloxane) comprises polydimethylsiloxane.

10. The carbonic anhydrase xerogel particle of claim 1 wherein the poly(silicone) is silanol-terminated.

11. The carbonic anhydrase xerogel particle of claim 1 comprising an alkoxysilane of tetramethyl orthosilicate, a poly(silicone) of a silanol-terminated poly(dimethyl siloxane), a hydrophilic additive of a crown ether, and a hydrophobic organotrialkoxy silane of isobutyl trimethoxysilane, nonafluorohexyl trimethoxysilane, or a combination thereof.

12. (canceled)

13. A carbonic anhydrase xerogel particle comprising carbonic anhydrase entrapped within a xerogel particle, the carbonic anhydrase xerogel particle being derived from a sol comprising (i) an alkoxy silane or an organotrialkoxy silane or metasilicate, (ii) a poly(silicone), and (iii) carbonic anhydrase wherein the carbonic anhydrase xerogel particle is further modified by a hydrophobic or hydrophilic modifier.

14-20. (canceled)

21. The carbonic anhydrase xerogel particle of claim 1 wherein the particle has a size from about 1 μm to about 250 μm.

22. The carbonic anhydrase xerogel particle of claim 1 wherein the sol is reacted with a catalyst.

23-30. (canceled)

31. The carbonic anhydrase xerogel particle of claim 1 wherein the hydrophilic additive is poly(vinyl alcohol), poly(ethylene oxide), a quaternary ammonium polymer, a crown ether, a cyclodextrin, a surfactant, hexadecyltrimethylammonium bromide, poly(1-methyl-4-vinylpyridinium bromide), poly(acrylamide-methacryloxyethyltrimethylammonium bromide), or a combination thereof.

32. The carbonic anhydrase xerogel particle of claim 31 wherein the hydrophilic additive comprises hexadecyltrimethylammonium bromide.

33. The carbonic anhydrase xerogel particle of claim 31 wherein the crown ether is 12-crown-4; 1,7-diaza-12-crown-4; 1,4,8,11-tetrathiacyclotetradecane; 1,4,8,12-tetraazacyclopentadecane; 15-crown-5; 18-crown-6; dibenzo-18-crown-6; dicyclohexano-18-crown-6; (18-crown-6)-2,3,11,12-tetracarboxylic acid; 1-aza-18-crown-6; diaza-18-crown-6; or a combination thereof.

34. The carbonic anhydrase xerogel particle of claim 31 wherein the crown ether comprises 18-crown-6.

35. The carbonic anhydrase xerogel particle of claim 1 wherein the sol further comprises a surfactant and the surfactant comprises cetyltrimethylammonium bromide (CTAB).

36-38. (canceled)

39. A process for removing CO₂ from a CO₂-containing gas, the process comprising
contacting a liquid with a CO₂-containing gas; and contacting the CO₂ in the liquid with the carbonic anhydrase xerogel particle of claim 1 to catalyze hydration of the CO₂ and form a treated liquid comprising hydrogen ions and bicarbonate ions.

40-61. (canceled)
